(19) United States
(12) Patent Application Publication
(10) Pub. No.: US 2026/0053899 A1
(43) Pub. Date: Feb. 26, 2026

(54) BIOMARKERS AND IMMUNOGENIC COMPOSITIONS FOR FILARIAL PARASITES

(71) Applicants: New York Blood Center, Inc., New York, NY (US); The United States of America, as Represented by the Secretary, Department of Health & Human service, Bethesda, MD (US)

(72) Inventors: Sara Lustigman, New York, NY (US); Thomas B. Nutman, Bethesda, MD (US); Sasisekhar Bennuru, Bethesda, MD (US)

(21) Appl. No.: 19/185,808

(22) Filed: Apr. 22, 2025

Related U.S. Application Data
(62) Division of application No. 18/173,288, filed on Feb. 23, 2023, now Pat. No. 12,311,016.
Application No. 18/173,288 is a division of application No. 17/076,616, filed on Oct. 21, 2020, now Pat. No. 12,251,432.
Application No. 17/076,616 is a division of application No. 16/090,013, now abandoned.
(60) Provisional application No. 62/317,243, filed on Apr. 1, 2016.

(51) Int. Cl. A61K 39/00; A61P 33/10; C07K 14/435; C07K 16/18; G01N 33/53

(52) U.S. Cl. CPC A61K 39/0003; A61P 33/10; C07K 14/4354; C07K 16/18; G01N 33/5308; A61K 2039/552; A61K 2039/55505; A61K 2039/6031; A61K 2039/70

(57) ABSTRACT

Disclosed herein are immunogenic compositions for preventing or treating infection with filarial parasites and biomarkers for diagnosing infection with filarial parasites.

### CROSS-REFERENCE TO RELATED APPLICATIONS

The present application is a divisional application of U.S. patent application Ser. No. 18/173,288, filed Feb. 23, 2023, which is a divisional application of U.S. patent application Ser. No. 17/076,616, filed Oct. 21, 2020, now U.S. Pat. No. 12,251,432, which is a divisional application of U.S. patent application Ser. No. 16/090,013 filed Sep. 28, 2018, which is a 35 U.S.C. § 371 national phase entry of PCT/US2017/025554, filed Mar. 31, 2017, which claims the benefit of U.S. Provisional Patent Application No. 62/317,243, filed Apr. 1, 2016, the entire contents of all of which are incorporated by reference herein.

### STATEMENT REGARDING FEDERALLY SPONSORED RESEARCH

This invention was made in part with government support under Grant/Contract Number AI42328 awarded by the Division of Microbiology and Infectious Diseases, National Institute of Allergy and Infectious Diseases, National Institutes of Health; the Division of Intramural Research (DIR) of the National Institute of Allergy and Infectious Diseases, National Institutes of Health. The government has certain rights in the invention.

### SEQUENCE LISTING

This application contains a Sequence Listing that has been submitted electronically as an XML file named 58434-0003004_SL_ST26.xml. The XML file, created on Apr. 22, 2025, is 72,516 bytes in size. The material in the XML file is hereby incorporated by reference in its entirety.

### BACKGROUND

Onchocerciasis (river blindness) is a neglected tropical disease, caused by infection with Onchocerca volvulus, that has been targeted for control and elimination through mass drug administration (MDA) of ivermectin that ultimately interrupts transmission. The ultimate success of MDA for onchocerciasis will largely depend on additional tools (macrofilaricidal drugs, vaccines, sensitive diagnostic biomarkers) that in turn rely on a comprehensive understanding of the biology of O. volvulus and the O. volvulus-human host interaction.

Because of the genetic similarity between O. volvulus and Dirofilaria immitis, the causative agent of heartworm in dogs, it is expected that the D. immitis orthologs of protective O. volvulus proteins will provide protection in dogs against infection with D. immitis as well. Vaccination of ‘at risk’ dogs is an increasingly important activity as dogs are becoming resistant to ivermectin, the current prophylactic drug for canine heartworm.

### SUMMARY

The present disclosure relates to immunogenic compositions for preventing or treating infection with filarial parasites and biomarkers for diagnosing infection with filarial parasites.

Thus, disclosed herein are immunogenic compositions for preventing or treating infection with a filarial parasite, wherein the filarial parasite is Onchocerca volvulus, and wherein the immunogenic composition comprises at least one filarial parasite protein having at least 85%, 90%, 95%, or 98% sequence identity to the full length mature protein of OVOC8619 (SEQ ID NO:16), OVOC7083 (SEQ ID NO:17), OVOC4111 (SEQ ID NO:18), OVOC1808 (SEQ ID NO:19), OVOC11598 (SEQ ID NO:20), OVOC3901 (SEQ ID NO:21), OVOC10819 (SEQ ID NO:22), OVOC5395 (SEQ ID NO:23), OVOC12235 (SEQ ID NO:24), OVOC7908 (SEQ ID NO:25), OVOC7430 (SEQ ID NO:26), OVOC8936 (SEQ ID NO:27), OVOC5806 (SEQ ID NO:28), OVOC4665 (SEQ ID NO:29), or OVOC8227 (SEQ ID NO:30).

Also disclosed herein are immunogenic compositions for preventing infection with a filarial parasite, wherein the filarial parasite is Dirofilaria immitis, and wherein the immunogenic composition comprises at least one filarial parasite mature protein having at least 85%, 90%, 95%, or 98% sequence identity to the full length of OVOC8619 (SEQ ID NO:16), OVOC7083 (SEQ ID NO:17), OVOC4111 (SEQ ID NO:18), OVOC1808 (SEQ ID NO:19), OVOC11598 (SEQ ID NO:20), OVOC3901 (SEQ ID NO:21), OVOC10819 (SEQ ID NO:22), OVOC5395 (SEQ ID NO:23), OVOC12235 (SEQ ID NO:24), OVOC7908 (SEQ ID NO:25), OVOC7430 (SEQ ID NO:26), OVOC8936 (SEQ ID NO:27), OVOC5806 (SEQ ID NO:28), OVOC4665 (SEQ ID NO:29), OVOC8227 (SEQ ID NO:30), OVOC9988 (SEQ ID NO:31), or OVOC4230 (SEQ ID NO:32), or an ortholog thereof. In some embodiments, the ortholog comprises a filarial parasite protein having at least 85%, 90%, 95%, or 98% sequence identity to the full length of one of SEQ ID NOs:33-49.

In some embodiments, an immunogenic composition further comprises an adjuvant. In certain embodiments, the immunogenic composition comprises at least two, at least three, at least four, at least five, at least six, at least seven, at least eight, at least nine, or at least ten filarial parasite proteins. In some embodiments, the at least two filarial parasite proteins are present in the immunogenic composition in a mixture. In certain embodiments, the at least two filarial parasite proteins are present in the immunogenic composition as a fusion protein comprising the amino acid sequences of the at least two filarial parasite proteins. In some embodiments, the fusion protein optionally further comprises at least one linker sequence separating the at least two filarial parasite amino acid sequences.

Also disclosed herein are methods of preventing infection with, or transmission of, O. volvulus, the method comprising administering an immunogenic composition disclosed herein to a subject in need thereof, wherein the immunogenic composition prevents the infection or prevents transmission of the infection to another subject. In some embodiments, the immunogenic composition further includes an adjuvant.

In some embodiments, the immunogenic composition is administered to a subject at risk of O. volvulus infection, and the administration prevents infection with O. volvulus and/or prevents transmission of O. volvulus. In some embodiments, the subject is a human.

Also disclosed herein are methods of preventing an infection with D. immitis, the method comprising administering an immunogenic composition disclosed herein to a canine subject in need thereof, wherein the immunogenic composition prevents the infection.

In some embodiments, the immunogenic composition is administered to a subject at risk of D. immitis infection, and the administration prevents infection with D. immitis.

Also disclosed herein are methods of detecting infection with O. volvulus, comprising identifying in a specimen from a subject at least one filarial full length mature protein having at least 85%, 90%, 95%, or 98% sequence identity to OVOC10469 (SEQ ID NO:1), OVOC11950 (SEQ ID NO:2), OVOC10602 (SEQ ID NO:3), OVOC3261 (SEQ ID NO:4), OVOC5127 (SEQ ID NO:5), OVOC8491 (SEQ ID NO:6), OVOC6759 (SEQ ID NO:7), OVOC451 (SEQ ID NO:8), OVOC12329 (SEQ ID NO:9), OVOC3337 (SEQ ID NO:10), OVOC10264 (SEQ ID NO:11), OVOC4230 (SEQ ID NO:12), OVOC8422 (SEQ ID NO:14), OVOC6395 (SEQ ID NO:15), or OVOC10384 (SEQ ID NO:13) or an immunoreactive fragment thereof.

Also disclosed herein are methods of detecting infection with O. volvulus, comprising identifying in the blood of a subject, antibodies to at least one filarial protein having at least 85%, 90%, 95%, or 98% sequence identity to the full length mature protein of OVOC10469 (SEQ ID NO:1), OVOC11950 (SEQ ID NO:2), OVOC10602 (SEQ ID NO:3), OVOC3261 (SEQ ID NO:4), OVOC5127 (SEQ ID NO:5), OVOC8491 (SEQ ID NO:6), OVOC6759 (SEQ ID NO:7), OVOC451 (SEQ ID NO:8), OVOC12329 (SEQ ID NO:9), OVOC3337 (SEQ ID NO:10), OVOC10264 (SEQ ID NO:11), OVOC4230 (SEQ ID NO:12), OVOC10384 (SEQ ID NO:13), OVOC8422 (SEQ ID NO:14), OVOC9988 (SEQ ID NO:31), or OVOC6395 (SEQ ID NO:15), or an immunoreactive fragment thereof.

In certain embodiments, the immunoreactive fragment is OVOC10469_Pep2 (SEQ ID NO:51), OVOC3261_Pep1 (SEQ ID NO:52), OVOC3261_Pep3 (SEQ ID NO:53), OVOC10469_Pep1 (SEQ ID NO:54), OVOC10469_Pep3 (SEQ ID NO:55), OVOC3261_Pep2 (SEQ ID NO:56), OVOC5127_Pep1 (SEQ ID NO:57), OVOC5127_Pep2 (SEQ ID NO:58), OVOC5127_Pep4, (SEQ ID NO:59), OVOC5127_Pep5 (SEQ ID NO:60), or OVOC5127_PepX (SEQ ID NO:61).

In certain embodiments, the specimen comprises blood, a skin biopsy, or urine.

In some embodiments, the method comprises at least two, at least three, at least four, at least five, at least six, at least seven, at least eight, at least nine, or at least ten filarial parasite proteins. In some embodiments, the at least two filarial parasite proteins comprise Ov16 and OVOC3261. In some embodiments, the at least four filarial parasite proteins comprise Ov16, OVOC3261, OVOC10469, and OVOC5127.

In some embodiments, the filarial protein or antibody to the filarial protein are detected by a method selected from the group consisting of ELISA, dipstick tests, lateral flow, microfluidic devices, luciferase immunoprecipitation systems, luminex, multiplex-formats, polymerase chain reaction, and microarrays.

### BRIEF DESCRIPTION OF THE DRAWINGS

FIG. 1A-B depicts an overview of transcriptome and proteome of Onchocerca volvulus. FIG. 1A depicts the number of O. volvulus-specific genes (light gray) and proteins (dark bars) identified across the stages by transcriptomic and proteomic analyses. ND denotes samples that were not analyzed. FIG. 1B depicts the number of Wolbachia (wOv) proteins identified across the O. volvulus stages.

FIG. 2A-B depicts immunoreactivity of disclosed O. volvulus proteins. FIG. 2A depicts a scatter plot of representative proteins with significant IgG4 reactivity in infected individuals, plotted as normalized intensity. FIG. 2B depicts scatter plots of representative proteins with significant IgG1, IgG3 and IgE responses in putatively immune individuals, plotted as ratios to normal sera. The four columns of dataform each protein are, from left to right: Putatively Immune; Normal; Infected individuals; Endemic Normal. P values are represented by *(p≤0.05), **(p≤0.01), ***(p≤0.001), ****(p≤0.001).

FIG. 3A-H depicts the sensitivity of the biomarkers for O. volvulus infection. FIG. 3A: Ov16; FIG. 3B: OVOC10469; FIG. 3C: OVOC3261, FIG. 3D: OVOC5127; FIG. 3E: Ov16; FIG. 3F: Ov16+OVOC10469; FIG. 3G: Ov16+OVOC10469+OVOC3261. FIG. 3H depicts the positivity (in black) for each protein based on ROC (receiver operating curves) values. The false negatives for Ov16 (gray, boxed) can be picked up using combination of the proteins (OVOC10469, OVOC3261 and OVOC5127). The white denotes samples not assayed for that protein.

FIG. 4A-C depicts the effect of immunization with a single vaccine antigen expressed by either Escherichia coli or Pichia pastoris on the development of protective immunity to O. volvulus larvae in mice. FIG. 4A: Ov-103; FIG. 4B: Ov-RAL-2; FIG. 4C: Ov-CPI-2M. Each dot represents larval recovery from an individual animal. Data presented are mean±S.D. Asterisk represents statistical difference in larval recoveries; P<0.05.

FIG. 5A-B depicts the effect of immunization with fusion antigens on the development of protective immunity to O. volvulus larvae in mice. FIG. 5A depicts Ov-RAL-2/103 fusion protein expressed in E. coli and P. pastoris expressed protein. FIG. 5B depicts Ov-RAL-2/CPI-2M expressed in E. coli. Each dot represents larval recovery from an individual animal. Data presented are mean±S.D. Asterisk represents statistical difference in larval recoveries; P<0.05.

FIG. 6 depicts the comparative effect of immunization with concurrent injections of O. volvulus Ov-103 (expressed in P. pastoris), Ov-RAL-2 (expressed in E. coli) and Ov-CPI-2M (expressed in E. coli) compared with immunization with the combined fusion antigen Ov-RAL-2/103/CPI-2M (expressed in E. coli). Each dot represents larval recovery from an individual animal. Data presented are mean±S.D. Asterisk represents statistical difference in larval recoveries; P<0.05.

### DETAILED DESCRIPTION

Onchocerciasis, or river blindness, caused by infection with Onchocerca volvulus, is a neglected tropical disease (NTD) that is associated with significant morbidity and disability in the 17 million people estimated to be infected. Infection leads to severe and disfiguring skin disease, lymphadenopathy and visual impairment (including blindness). Onchocerciasis was the first NTD targeted for control in 1974 by the World Health Organization (WHO) and is now one of the six NTDs targeted for elimination. Elimination efforts for O. volvulus are presently aimed by controlling transmission through ivermectin-based mass drug administration (MDA) programs, that have largely eliminated onchocerciasis in the Americas and that have made significant progress toward that goal in some regions of Africa. However, according to a new WHO evaluation, elimination would require an estimated 1.30 billion ivermectin treatments, lasting until 2045, and a recent report has suggested that onchocerciasis cannot solely be eliminated through MDA with ivermectin. Moreover, ivermectin is contraindicated in areas of marked co-endemicity with Loa loa, where the risk of severe adverse events is associated with high levels of circulating Loa loa microfilariae (mf). Furthermore, the potential for ivermectin resistance, the lack of macrofilaricidal activity by ivermectin, and the long timeline (>20 years) for transmission interruption has prompted research into the development of new tools (macrofilaricidal drugs, diagnostics, vaccines, etc.), the basis of which relies on a fundamental understanding of the parasite biology.

Humans are the only definitive host for O. volvulus. Because there are no existing small animal models for propagating the life cycle of O. volvulus, approaches that require sufficient amounts of stage-specific parasite material have been difficult, as the adult parasites must be obtained surgically from subcutaneous nodules and microfilariae from human skin. Moreover, the larval stages must be obtained from the infected blackflies—a process that to date requires feeding of newly hatched naïve black flies on infected microfiladermic humans. Nevertheless, using parasite material from most of the life cycle stages, a comprehensive profile of the stage-specific transcriptomes and proteomes of O. volvulus has been developed. Systematic comparisons across the parasite stages and across related nematodes and ‘immunomics” has enabled the identification of novel vaccine and diagnostic candidates.

Systems biology aims at understanding biological processes by integrating various omic's data. Compared to transcriptomic data, attaining complete coverage at the protein level is fraught with technological limitations as well as the dynamic nature of any proteome. Although a difference in transcript (RNA) and protein recovery from the various stages is expected, normalization (RPKM and spectral abundance) provides provisional evidence for relative abundance of any particular gene/protein in a given stage. Using a combination of transcriptomic and proteomic analyses comprehensive stage-specific analyses of O. volvulus was undertaken. This dataset provides an in-depth resource for understanding and analyzing the biological pathways that are critical for the development of the various stages of the parasite in the vector and human hosts, host-O. volvulus interaction, and for the identification of novel biomarkers and targets for interventions.

Natural immunity against O. volvulus can be acquired in a few individuals within affected populations; these individuals are known as putatively immune and exhibit protective immune response against L3 larvae, suggesting that E/S products released by molting larvae and/or surface proteins of L3 larvae are an important source of protective antigens. The identification of proteins that are highly expressed by the mf and that are specifically recognized by sera from protected individuals who never developed a clinically relevant infection also suggests other suitable vaccine candidates. The identification of O. volvulus-unique proteins that are adult and/or mf stage-specific identified by infected individuals, provided additional novel biomarkers needed for better mapping the prevalence of infection and for post-control surveillance.

As used herein the term “transcriptome” refers to the full range of messenger RNA, or mRNA, molecules expressed by an organism at a certain time.

As used herein, the term “proteome” refers to the entire set of proteins expressed by a genome, cell, tissue, or organism at a certain time.

The life cycle of O. volvulus includes the following stages: nodular microfilariae (NodMF), skin microfilariae (SknMF), embryos (OvEMB), larva L1 (OvL1), larva L2 (OvL2), larva L3 (OvL3), molting L3s (L3 Day 1 and L3 Day 3), larva L4 (OvL4), adult male (OvAM), and adult female (OvAF).

Analyses of transcript levels or protein abundance for each of the stages identified 363 proteins that were found as core elements by having been present across all somatic stages. Functionally, proteins involved in metabolism, cytoskeletal processes and protein modification comprised more than 50% of these core genes. Proteins shared between OvEMB and OvAF are likely to play a role in embryogenesis. Similarly, proteins identified exclusively during the L3 to L4 transition highlight the machinery required during the developmental molt, and possibly adaptation to the human host environment. Based on C. elegans RNAi data, O. volvulus homologs of C. elegans that exhibit phenotypes of embryonic lethality (EMB), larval arrest (LVA), larval lethal (LVL), molting defective (MLT), or lethal (LET) were observed to be clustered not only in embryos, microfilariae (and thereby adult females), and L3 larval stages but also in adult males. This could either be due to C. elegans being primarily a hermaphroditic organism or to differences between gene families of parasitic and free-living nematodes.

Similarly, the O. volvulus genome encodes orthologues of the most critical genes essential for molting (based on C. elegans), orthologues that appear to be highly expressed during the in vitro molting process of the L3 larvae. However, it also highlights other proteins, some of which have already been shown to be essential for molting and/or other developmental processes of filarial parasites. For example, embryogenesis and molting in filarial parasites is dependent on the activity of cathepsin L-like cysteine proteases (CPLs).

Establishment of infection in humans depends on the successful molt from L3 to L4 larvae and subsequent development into adults. During molting, CPLs are stored in the glandular esophagus and their release during molting helps breakdown the old cuticle and drives synthesis of a new cuticle by processing the pro-proteins. Comparative analyses suggest an expansion of CPL-like enzymes in the O. volvulus genome. Significant transcriptional regulation of CPL and C PZ molecules was observed in L2 and L3 larvae compared to other stages. Inferring from Brugia malayi, a related filiarial parasite, these enzymes are probably needed for the L2 to L3 molt in the black fly. Interestingly, the GO gene categories of nucleotide binding (GO:0000166), molecular function (GO:0003674), and phosphoprotein phosphatase activity (GO:0004721), were the most represented categories of differentially expressed genes during L2/L3 and L3/L4 molting. Gene set enrichment analysis (GSEA) identified immunologically important classes of molecules as enriched in L3 larval stages, and a set of extracellular matrix-related genes distinct from the ones overexpressed in adult female worms. The collagens making up the cuticle are regulated by a number of factors, one of which is prolyl-4 hydroxylase, a family that is expanded in the O. volvulus genome, and that is expressed in a stage-specific manner.

In contrast to those gene families upregulated during development, nuclear hormone receptors (NHR), known to play an important role in other nematode developmental processes, are comparatively less expanded in O. volvulus but still appear to play a role in molting and embryogenesis, as seen in B. malayi. Indeed the O. volvulus ecdysone receptor (EcR, Accession No. OVOC9104) and NHR RXR (Accession No. OVOC2435) are upregulated during the L3 to L4 developmental molt. Furthermore GSEA indicate enrichment of OVOC351 and OVOC353 (other potential NHRs) in adult female worms (p-value<0.0001, FDR<1%). Similarly, the orthologues of the C. elegans NHRs—nhr-6 (OVOC8200), nhr-23 (OVOC464), nhr-25 (OVOC2839), nhr-41 (OVOC4741) and nhr-85 (OVOC827)—known to be involved in molting and metamorphosis, are present in the O. volvulus genome and detected as transcripts or proteins during the in vitro molting of L3 to L4. In addition, NHRs implicated in neural differentiation (OVOC635, OVOC3708) and sex determination (OVOC5276) were upregulated in the molting stages reflecting their probable role in molting, growth, and sex determination.

Protein OVOC2265 has a rather unique expression profile in the nodular microfilariae (mf) that corresponded with the proteome of embryonic stages. Among the embryo-enriched transcripts and proteins, OVOC11613 (immunodominant antigen or major antigen), and OVOC9384 (Oveg1) have been shown to be related to embryogenesis as well.

The O. volvulus sequences disclosed here correspond to the WS245 release of the genome by WormBase. The D. immitis sequences disclosed herein correspond to the WPBS1 release of the genome by WormBase. Subsequent genome releases by WormBase may have nucleotide or amino acid revisions.

### I. Biomarkers

Thus provided herein are biomarkers for infection with a filarial parasite. In certain embodiments, the filarial parasite is O. volvulus.

In certain embodiments, if the filarial parasite is O. volvulus, the biomarker is a protein having at least 85%, at least 90%, at least 95%, or at least 98% sequence identity to the full length mature protein of OVOC10469 (SEQ ID NO:1), OVOC11950 (SEQ ID NO:2), OVOC10602 (SEQ ID NO:3), OVOC3261 (SEQ ID NO:4), OVOC5127 (SEQ ID NO:5), OVOC8491 (SEQ ID NO:6), OVOC6759 (SEQ ID NO:7), OVOC451 (SEQ ID NO:8), OVOC12329 (SEQ ID NO:9), OVOC3337 (SEQ ID NO:10), OVOC10264 (SEQ ID NO:11), OVOC4230 (SEQ ID NO:12), OVOC10384 (SEQ ID NO:13), OVOC8422 (SEQ ID NO:14), or OVOC6395 (SEQ ID NO:15).

In addition, the biomarker can also include proteins and peptides sharing a sequence identity or substantial sequence identity to the biomarker proteins provided herein.

As used herein, “sequence identity” or “identity” in the context of two protein or peptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art.

The term “substantial identity” in the context of a protein or peptide indicates that a protein or peptide comprises a sequence with at least 85%, 86%, 87%, 88%, 89%, 90%, 91%, 92%, 93%, or 94%, or even 95%, 96%, 97%, 98%, or 99% sequence identity to the reference sequence over a specified comparison window. In certain embodiments, optimal alignment is conducted using the homology alignment algorithm of Needleman and Wunsch (Needleman and Wunsch, JMB, 48:443, 1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, also provided herein are proteins and peptides that are substantially identical to the proteins and peptides presented herein.

In certain embodiments, the term “sequence identity” refers to identity across the entire amino acid sequence of one of SEQ ID NOs:1-66 but can include proteins or peptides which have additional amino acids at the C-terminus or N-terminus of the protein or peptide and which have at least 85%, at least 90%, at least 95%, or at least 98% sequence identity to the portion of the sequence which is the same length as the disclosed sequences.

Accordingly, some embodiments disclosed herein comprise a method of diagnosing an infection with a filarial parasite comprising: (a) providing a blood sample from at least one subject suspected of having a filarial parasite infection; and (b) contacting the sample with at least one protein selected from OVOC10469 (SEQ ID NO:1), OVOC11950 (SEQ ID NO:2), OVOC10602 (SEQ ID NO:3), OVOC3261 (SEQ ID NO:4), OVOC5127 (SEQ ID NO:5), OVOC8491 (SEQ ID NO:6), OVOC6759 (SEQ ID NO:7), OVOC451 (SEQ ID NO:8), OVOC12329 (SEQ ID NO:9), OVOC3337 (SEQ ID NO:10), OVOC10264 (SEQ ID NO:11), OVOC4230 (SEQ ID NO:12), OVOC8422 (SEQ ID NO:14), OVOC6395 (SEQ ID NO:15) or OVOC10384 (SEQ ID NO:13); wherein if the sample contains specific antibodies which bind to the at least one protein, the subject has an active filarial parasite infection. In certain embodiments, the method includes contacting the sample with at least two, at least three, at least four, at least five, at least six, at least seven, at least eight, at least nine, or at least ten filarial parasite proteins.

Additionally, some embodiments disclosed herein comprise a method of diagnosing an infection with a filarial parasite comprising: (a) providing a tissue or fluid sample from at least one subject suspected of having an filarial parasite infection; (b) providing a binding agent which binds to at least one filarial parasite-associated protein selected from OVOC10469 (SEQ ID NO:1), OVOC11950 (SEQ ID NO:2), OVOC10602 (SEQ ID NO:3), OVOC3261 (SEQ ID NO:4), OVOC5127 (SEQ ID NO:5), OVOC8491 (SEQ ID NO:6), OVOC6759 (SEQ ID NO:7), OVOC451 (SEQ ID NO:8), OVOC12329 (SEQ ID NO:9), OVOC3337 (SEQ ID NO:10), OVOC10264 (SEQ ID NO:11), OVOC4230 (SEQ ID NO:12), OVOC8422 (SEQ ID NO:14), OVOC6395 (SEQ ID NO:15) OVOC10384 (SEQ ID NO:13); and (c) detecting the proteins, individually and/or in combination, associated with the filarial parasite infection in the subject and contained in the sample; wherein if the sample contains at least one filarial parasite-associated protein, the subject has an active filarial parasite infection. In certain embodiments, the fluid sample is urine, blood, serum, plasma, or a skin biopsy. In some embodiments, the method includes detecting at least two, at least three, at least four, at least five, at least six, at least seven, at least eight, at least nine, or at least ten filarial parasite proteins.

Also disclosed herein are immunoreactive fragments of filarial parasite proteins which can be used in the methods disclosed herein. The immunoreactive fragments include, but are not limited to, OVOC10469_Pep2 (SEQ ID NO:51), OVOC3261_Pep1 (SEQ ID NO:52), OVOC3261_Pep3 (SEQ ID NO:53), OVOC10469_Pep1 (SEQ ID NO:54), OVOC10469_Pep3 (SEQ ID NO:55), OVOC3261_Pep2 (SEQ ID NO:56), OVOC5127_Pep1 (SEQ ID NO:57), OVOC5127_Pep2 (SEQ ID NO:58), OVOC5127_Pep4, (SEQ ID NO:59), OVOC5127_Pep5 (SEQ ID NO:60), and OVOC5127_PepX (SEQ ID NO:61).

Ideally, several methods, including ELISA, dipstick tests, lateral flow, microfluidic devices, luciferase immunoprecipitation systems (LIPS), and microarrays can be used to detect filarial parasite-associated biomarkers in patients with filarial parasite infections.

ELISA is a widely used method for the detection of specific antibodies and proteins in a biological sample. It involves the immobilization of an antibody (primary antibody), or an antigen, to a solid support, such as plastic microplates, and detecting binding of components of a patient sample to the immobilized antibody or antigen, followed by the addition of secondary antibody or antibodies, the latter usually being conjugated to a detectable moiety in order to facilitate measurement.

Hence, according to some embodiments, the immune affinity procedure may be an ELISA immunoassay selected from the group consisting of direct enzyme-linked immunosorbent assays, indirect enzyme-linked immunosorbent assays, direct sandwich enzyme-linked immunosorbent assays, indirect sandwich enzyme-linked immunosorbent assays, and competitive enzyme-linked immunosorbent assays.

In one embodiment, detection is effected through capture ELISA. Capture ELISA (also known as “sandwich” ELISA) is a sensitive assay to quantify picogram to microgram quantities of substances such as hormones, cell signaling chemicals, infectious disease antigens and cytokines. This type of ELI SA is particularly sought after when the substance to be analyzed may be too dilute to bind to the microtiter plate (such as a protein in a cell culture supernatant) or does not bind well to plastics (such as a small organic molecule). Optimal dilutions for the capture antibody, samples, controls, and detecting antibodies as well as incubation times are determined empirically and may require extensive titration. Ideally, one would use an enzyme-labeled detection antibody. However, if the detection antibody is unlabeled, the secondary antibody should not cross-react with either the coating antibody or the sample. Optimally, the appropriate negative and positive controls should also be included.

Detection of the biomarkers, or of any fragment or derivative thereof, may be performed using antibodies specific to said biomarkers. These antibodies may be labeled directly or indirectly by a detectable moiety.

As used herein in the specification, the term “detectable moiety” refers to any element, molecule, or a portion thereof, the presence, absence or level of which may be monitored directly or indirectly. One example includes radioactive isotopes. Other examples include enzymes which can catalyze color or light emitting (luminescence) reactions, fluorophores, and gold or magnetic labels. The detection of the detectable moiety can be direct provided that the detectable moiety is itself detectable (i.e. can be directly visualized or measured), such as, for example, in the case of fluorophores. Alternatively, the detection of the detectable moiety can be indirect. In the latter case, a second moiety that reacts with the detectable moiety, itself being directly detectable is preferably employed. The detectable moiety may be inherent to the antibody. For example, the constant region of an antibody can serve as an indirect detectable moiety to which a secondary antibody having a direct detectable moiety can specifically bind.

Thus, secondary antibodies are particular suitable means for the detection of the anti-biomarker antibody. This secondary antibody may be itself conjugated to a detectable moiety. One of the ways in which an antibody can be detectably labeled is by linking the same to an enzyme. The enzyme, in turn, when exposed to an appropriate substrate, will react with the substrate in such a manner as to allow its detection, for example, by producing a chemical moiety, which can be detected, for example, by spectrophotometric, fluorometric, or by visual means. Enzymes which may be used to label the antibody include, but are not limited to, horseradish peroxidase, alkaline phosphatase, malate dehydrogenase, staphylococcal nuclease, 6-5-steroid isomerase, yeast alcohol dehydrogenase, α-glycerophosphate dehydrogenase, triose phosphate isomerase, asparaginase, glucose oxidase, beta-galactosidase, ribonuclease, urease, catalase, glucose-6-phosphate dehydrogenase, glucoamylase and acetylcholinesterase, or any other enzyme which can be conjugated to an antibody and its reaction with a substrate, measured or detected.

The detection may be accomplished by colorimetric methods, which employ a chromogenic substrate for the enzyme. Detection may also be accomplished by visual comparison of the extent of enzymatic reaction of a substrate in comparison with similarly prepared standards.

The solid support to which the first antibody is bound may be any water-insoluble, water-insuspensible, solid support. Examples of suitable solid support include, but are not limited to, large beads (e.g., of polystyrene), filter paper, slides, chips, test tubes, and microtiter plates. The first antibody may be bound to the solid support by covalent bonds or by adsorption. The advantage in using a solid support is that no centrifugation step is needed for the separation of the solid and liquid phase.

The solid support mentioned above may include polymers, such as polystyrene, agarose, SEPHAROSE®, cellulose, glass beads and magnetizable particles of cellulose or other polymers. The solid-support can be in the form of large or small beads or particles, tubes, plates, slides, chips or other forms.

As a solid support, a test tube, the inner walls of a test tube or a microtiter plate are coated with a first antibody, e.g., antibodies specific to a peptide or protein disclosed herein, or of any fragment or derivative thereof.

In a further embodiment, dipstick assays may be used to detect filarial parasite biomarkers. Dipstick assays use the lateral flow format, wherein capture antibodies are striped or banded onto nitrocellulose membrane and a wicking pad draws the sample up through the dipstick, whereby the filarial parasite biomarkers interact with a filarial parasite biomarker antibody, or combination of antibodies. Other antibodies specific to filarial parasites, or other proteins of interest may be included. Subsequent analysis of enzyme activity and protein quantity may be done using standard methods known to a person skilled in the art, or as discussed above regarding ELISAs.

In another preferred embodiment, microfluidic devices, which may also be referred to as “lab-on-a-chip” systems, biomedical micro-electro-mechanical systems (bioMEMs), or multicomponent integrated systems, may be used for detecting filarial parasite biomarkers. Such systems miniaturize and compartmentalize processes that allow for detection of filarial parasite biomarkers, and other processes.

Array-based assays and bead-based assays may be used with microfluidic device. For example, a binding agent can be coupled to beads and the binding reaction between the beads and filarial parasite biomarker can be performed in a microfluidic device. Multiplexing, or detecting more than one filarial parasite biomarker at once, can also be performed using a microfluidic device. Different compartments can comprise different binding agents for different populations of filarial parasite biomarkers, where each population has a different bio-signature.

In another embodiment, microarrays are used to detect filarial parasite biomarkers. Microarrays are typically small, high throughput chips generally made of a solid support structure, typically glass slides, nitrocellulose, or microtiter plates. Generally, antibodies to specific biomarker are bound to the solid support; however, other molecules, such as, but not limited to other proteins, aptamers, DNA, RNA, sugars or lipids can be bound to the solid surface as well. Detection of the captured biomarker can also be accomplished as discussed above for ELISA detection.

In another further embodiment, recognition of filarial parasite specific biomarker is achieved through an immune affinity procedure such as Western blot, immuno-precipitation, FACS, biochip array, lateral flow, time resolved fluorometry, ECL procedures, luminex, LIPS, multiplex-immunoassay formats or any procedure based on immune recognition known to one of ordinary skill in the art.

### II. Immunogenic Compositions

Also provided herein are immunogenic compositions for preventing infection with, or preventing transmission of, a filarial parasite. In certain embodiments, the filarial parasite is O. volvulus. In other embodiments, the filarial parasite is D. immitis. As used herein, “preventing transmission of” refers to the inability of an infected subject, who has been immunized with an immunogenic disclosed herein, to transmit infectious parasites to another subject via an intermediate vector.

In certain embodiments, the filarial parasite is O. volvulus, and the immunogenic composition comprises as least one protein having at least 85%, at least 90%, at least 95%, or at least 98% sequence identity to the full length mature protein of OVOC8619 (SEQ ID NO:16), OVOC7083 (SEQ ID NO:17), OVOC4111 (SEQ ID NO:18), OVOC1808 (SEQ ID NO:19), OVOC11598 (SEQ ID NO:20), OVOC3901 (SEQ ID NO:21), OVOC10819 (SEQ ID NO:22), OVOC5395 (SEQ ID NO:23), OVOC12235 (SEQ ID NO:24), OVOC7908 (SEQ ID NO:25), OVOC7430 (SEQ ID NO:26), OVOC8936 (SEQ ID NO:27), OVOC5806 (SEQ ID NO:28), OVOC4665 (SEQ ID NO:29), OVOC8227 (SEQ ID NO:30), OVOC9988 (SEQ ID NO:31), OVOC4230 (SEQ ID NO:32), or an immunogenic fragment thereof, or a nucleic acid encoding the protein. In certain embodiments, the filarial parasite immunogenic composition for preventing infection with, or preventing transmission of, O. volvulus is not OVOC9988 (Ov-RAL-2), OVOC4230 (Ov-103), or OVOC7453 related (Ov-CPI-2M). In some embodiments, the immunogenic composition includes at least two, at least three, at least four, at least five, at least six, at least seven, at least eight, at least nine, or at least ten filarial parasite proteins. The immunogenic composition can comprise the filarial parasite proteins in a mixture or as fusion proteins which include sequences from two or more filarial parasite proteins assembled in a single polypeptide sequence. If multiple filarial proteins are assembled into a fusion protein, one or more linker sequences can be included.

In other embodiments, the filarial parasite is D. immitis, and the immunogenic composition comprises at least one protein having at least 85%, at least 90%, at least 95%, or at least 98% sequence identity to the full length mature protein of OVOC8619 (SEQ ID NO:16), OVOC7083 (SEQ ID NO:17), OVOC4111 (SEQ ID NO:18), OVOC1808 (SEQ ID NO:19), OVOC11598 (SEQ ID NO:20), OVOC3901 (SEQ ID NO:21), OVOC10819 (SEQ ID NO:22), OVOC5395 (SEQ ID NO:23), OVOC12235 (SEQ ID NO:24), OVOC7908 (SEQ ID NO:25), OVOC7430 (SEQ ID NO:26), OVOC8936 (SEQ ID NO:27), OVOC5806 (SEQ ID NO:28), OVOC4665 (SEQ ID NO:29), OVOC8227 (SEQ ID NO:30), OVOC9988 (SEQ ID NO:31), or OVOC4230 (SEQ ID NO:32), an ortholog thereof, or a nucleic acid encoding the protein or ortholog. In some embodiments, the ortholog comprises one of the proteins of Table 6, or an immunogenic fragment thereof. In some embodiments, the immunogenic composition includes at least two, at least three, at least four, at least five, at least six, at least seven, at least eight, at least nine, or at least ten filarial parasite proteins. The immunogenic composition can comprise the filarial parasite proteins in a mixture or as fusion proteins which include sequences from two or more filarial parasite proteins assembled in a single polypeptide sequence.

As used herein, the term “immunogenic composition” refers to a substance which induces a specific immune response against an immunogen (protein) in an individual who is in need of an immune response to the immunogen. As used herein the term “immunogen” refers to any substrate that elicits an immune response in a host. Thus, the disclosed immunogenic compositions comprising filarial parasite proteins are useful for inducing an immune response against a filarial parasite. In certain embodiments, the immune response is a protective immune response. In other embodiments, the immune response is a therapeutic immune response. A non-limiting example of an immunogenic composition is a vaccine.

In certain embodiments, the immunogenic composition comprises a protein disclosed herein along with additional sequences to enhance immunogenicity.

In certain embodiments, the immunogenic composition is a fusion protein which includes several filarial parasite proteins. In some embodiments, the immunogenic composition is a mixture of one or more filarial parasite proteins. In some embodiments, the immunogenic composition includes at least two, at least three, at least four, at least five, at least six, at least seven, at least eight, at least nine, or at least ten filarial parasite proteins.

Certain amino acid sequences disclosed herein may include a signal sequence. As is understood by a person of skill in the art, expressed filarial proteins useful as immunogenic composition or biomarkers disclosed herein will not include a signal sequence. Thus, in certain embodiments, the amino acid sequences are referred to as “mature” proteins, which are proteins without a signal sequence. In some embodiments, the protein sequence will be recited as less than the entire amino acid sequence disclosed herein to reflect the absence of the signal sequence.

The disclosed filarial parasite immunogenic compositions and proteins include conservative variants of the proteins and fusion proteins. A conservative variant refers to a peptide or protein that has at least one amino acid substituted by another amino acid, or an amino acid analog, that has at least one property similar to that of the original amino acid from an exemplary reference peptide. Examples of properties include, without limitation, similar size, topography, charge, hydrophobicity, hydrophilicity, lipophilicity, covalent-bonding capacity, hydrogen-bonding capacity, physicochemical property, or the like, or any combination thereof. A conservative substitution can be assessed by a variety of factors, such as, e.g., the physical properties of the amino acid being substituted (Table 1) or how the original amino acid would tolerate a substitution (Table 2). The selections of which amino acid can be substituted for another amino acid in a peptide disclosed herein are known to a person of ordinary skill in the art. A conservative variant can function in substantially the same manner as the exemplary reference peptide, and can be substituted for the exemplary reference peptide in any aspect of the present specification.

TABLE 1
Amino Acid Properties

| Property | Amino Acids |
|---|---|
| Aliphatic | G, A, I, L, M, P, V |
| Aromatic | F, H, W, Y |
| C-beta branched | I, V, T |
| Hydrophobic | C, F, I, L, M, V, W |
| Small polar | D, N, P |
| Small non-polar | A, C, G, S, T |
| Large polar | E, H, K, Q, R, W, Y |
| Large non-polar | F, I, L, M, V |
| Charged | D, E, H, K, R |
| Uncharged | C, S, T |
| Negative | D, E |
| Positive | H, K, R |
| Acidic | D, E |
| Basic | K, R |
| Amide | N, Q |

TABLE 2
Amino Acid Substitutions

| Amino | Favored | | |
|---|---|---|---|
| Acid | Substitution | Neutral Substitutions | Disfavored substitution |
| A | G, S, T | C, E, I, K, M, L, P, Q, R, V | D, F, H, N, Y, W |
| C | F, S, Y, W | A, H, I, M, L, T, V | D, E, G, K, N, P, Q, R |
| D | E, N | G, H, K, P, Q, R, S, T | A, C, I, L, |
| E | D, K, Q | A, H, N, P, R, S, T | C, F, G, I, L, M, V, W, Y |
| F | M, L, W, Y | C, I, V | A, D, E, G, H, K, N, P, Q, R, S, T |
| G | A, S | D, K, N, P, Q, R | C, E, F, H, I, L, M, T, V, W, Y |
| H | N, Y | C, D, E, K, Q, R, S, T, W | A, F, G, I, L, M, P, V |
| I | V, L, M | A, C, T, F, Y | D, E, G, H, K, N, P, Q, R, S, W |
| K | Q, E, R | A, D, G, H, M, N, P, S, T | C, F, I, L, V, W, Y |
| L | F, I, M, V | A, C, W, Y | D, E, G, H, K, N, P, Q, R, S, T |
| M | F, I, L, V | A, C, R, Q, K, T, W, Y | D, E, G, H, N, P, S |
| N | D, H, S | E, G, K, Q, R, T | A, C, F, I, L, M, P, V, W, Y |
| P | — | A, D, E, G, K, Q, R, S, T | C, F, H, I, L, M, N, V, W, Y |
| Q | E, K, R | A, D, G, H, M, N, P, S, T | C, F, I, L, V, W, Y |
| R | K, Q | A, D, E, G, H, M, N, P, S, T | C, F, I, L, V, W, Y |
| S | A, N, T | C, D, E, G, H, K, P, Q, R, T | F, I, L, M, V, W, Y |
| T | S | A, C, D, E, H, I, K, M, N, P, Q, | F, G, L, W, Y |
| | | R, V | |
| V | I, L, M | A, C, F, T, Y | D, E, G, H, K, N, P, Q, R, S, W |
| W | F, Y | H, L, M | A, C, D, E, G, I, K, N, P, Q, R, S, |
| | | | T, V |
| Y | F, H, W | C, I, L, M, V | A, D, E, G, K, N, P, Q, R, S, T |

Matthew J. Betts and Robert, B. Russell, Amino Acid Properties and Consequences of Substitutions, pp. 289-316, In Bioinformatics for Geneticists, (eds Michael R. Barnes, Ian C. Gray, Wiley, 2003).

A filarial parasite immunogenic composition can also comprise conservative variants to the disclosed proteins or fusion proteins. In aspects of this embodiment, a conservative variant of a filarial parasite protein or fusion protein can be, for example, an amino acid sequence having at least 75%, at least 80%, at least 85%, at least 90%, at least 95%, at least 97%, at least 98%, or at least 99% amino acid sequence identity to the filarial parasite protein or fusion protein. In other aspects of this embodiment, a conservative variant of a filarial parasite protein or fusion protein can be, for example, an amino acid sequence having at most 75%, at most 80%, at most 85%, at most 90%, at most 95%, at most 97%, at most 98%, or at most 99% amino acid sequence identity to the filarial parasite protein or fusion protein.

In other embodiments, a conservative variant of a filarial parasite protein or fusion protein amino acid sequence can have, for example, 1, 2, 3, 4, 5, 6, 7, 8, 9, 10, 11, 12, 13, 14, 15, or more conservative substitutions, to the amino acid sequence of the filarial parasite protein or fusion protein. In other embodiments, a conservative variant of a filarial parasite protein or fusion protein amino acid sequence can be, for example, an amino acid sequence having at least 1, at least 2, at least 3, at least 4, at least 5, at least 6, at least 7, at least 8, at least 9, at least 10, at least 11, at least 12, at least 13, at least 14, or at least 15 conservative substitutions to the amino acid sequence of the filarial parasite protein or fusion protein. In yet other embodiments, a conservative variant of a filarial parasite protein or fusion protein amino acid sequence can be, for example, an amino acid sequence having at most 1, at most 2, at most 3, at most 4, at most 5, at most 6, at most 7, at most 8, at most 9, at most 10, at most 11, at most 12, at most 13, at most 14, or at most 15 conservative substitutions to the amino acid sequence of the filarial parasite protein or fusion protein. In further embodiments, a conservative variant of a filarial parasite protein or fusion protein amino acid sequence can be, for example, an amino acid sequence having from 1 to 15, 2 to 15, 3 to 15, 4 to 15, 5 to 15, 6 to 15, 7 to 15, 1 to 12, 2 to 12, 3 to 12, 4 to 12, 5 to 12, 6 to 12, 7 to 12, 1 to 10, 2 to 10, 3 to 10, 4 to 10, 5 to 10, 6 to 10, 7 to 10, 1 to 8, 2 to 8, 3 to 8, 4 to 8, 5 to 8, 6 to 8, 1 to 6, 2 to 6, 3 to 6, 4 to 6, 1 to 4, 2 to 4, or 1 to 3 conservative substitutions to the amino acid sequence of the filarial parasite protein or fusion protein.

In certain embodiments, the immunogenic compositions further comprise or are administered with an adjuvant. Adjuvants suitable for use in animals include, but are not limited to, Freund's complete or incomplete adjuvants, Sigma Adjuvant System (SAS), and Ribi adjuvants. Adjuvants suitable for use in humans include, but are not limited to, MF59® (an oil-in-water emulsion adjuvant, Novartis AG); MONTANIDE® ISA 51 or 720 (a mineral oil-based or metabolizable oil-based adjuvant, SEPPIC); aluminum hydroxide, -phosphate, or -oxide; HAVLOGEN® (an acrylic acid polymer-based adjuvant, Intervet Inc.); polyacrylic acids; oil-in-water or water-in-oil emulsion based on, for example a mineral oil, such as BAYOL™ or MARCOL™ (Esso Imperial Oil Limited), or a vegetable oil such as vitamin E acetate; a saponin; CpG oligodeoxynucleotide adjuvants; or a glucagon-like peptide (GLP) adjuvant. However, components with adjuvant activity are widely known and, generally, any adjuvant may be utilized that does not adversely interfere with the efficacy or safety of the immunogenic composition.

Immunogenic compositions according to the various embodiments disclosed herein can be prepared and/or marketed in the form of a liquid, frozen suspension, or in a lyophilized form. Typically, vaccines and/or immunogenic compositions contain a pharmaceutically acceptable carrier or diluent customarily used for such compositions. Carriers include, but are not limited to, stabilizers, preservatives, and buffers. Suitable stabilizers are, for example SPGA, TWEEN® compositions (such as are available from A.G. Scientific, Inc.), carbohydrates (such as sorbitol, mannitol, starch, sucrose, dextran, glutamate, or glucose), proteins (such as dried milk serum, albumin, or casein), or degradation products thereof. Examples of suitable buffers include alkali metal phosphates. Suitable preservatives include thimerosal, merthiolate, and gentamicin. Diluents include water, aqueous buffer (such as buffered saline), alcohols, and polyols (such as glycerol).

Also disclosed herein are methods for inducing an immune response to a filarial parasite using the disclosed proteins. Generally, the vaccine and/or immunogenic composition may be administered subcutaneously, intradermally, submucosally, intranasally, or intramuscularly in an effective amount to prevent infection from the filarial parasite and/or treat an infection from the filarial parasite. An effective amount to prevent infection is an amount of immunizing protein that will induce immunity in the immunized animals against challenge by infective stage larvae or microfilariae such that infection is prevented or the severity is reduced. Immunity is defined herein as the induction of a significant higher level of protection in a subject after immunization compared to an unimmunized group. An effective amount to treat an infection is an amount of immunizing protein that induces an appropriate immune response against filarial parasite such that severity of the infection is reduced.

Protective immune responses can include humoral immune responses and cellular immune responses. Protection against filarial parasite is believed to be conferred through serum antibodies (humoral immune response) directed to the surface proteins and/or proteins secreted during the early development in the human host, probably through antibody-dependent cellular cytotoxicity (ADCC) and cell-mediated immune responses. Cellular immune responses are useful in protection against filarial parasite infection with CD4+ T cell responses of the Th1, Th2 and/or Th17 type being particularly important. Additionally, the disclosed proteins and/or immunogenic compositions can be administered using immunization schemes known by persons of ordinary skill in the art to induce protective immune responses. These include a single immunization or multiple immunizations in a prime-boost strategy. A boosting immunization can be administered at a time after the initial, prime, immunization that is days, weeks, months, or even years after the prime immunization. In certain embodiments, a boost immunization is administered 2 weeks, 1 month, 2 months, 3 months, 4 months, 5 months, or 6 months or more after the initial prime immunization. Additional multiple boost immunizations can be administered such as weekly, every other week, monthly, every other month, every third month, or more. In other embodiments, the boost immunization can be administered every 3 weeks, every 4 weeks, every 5 weeks, every 6 weeks, every 7 weeks, every 8 weeks, every 9 weeks, every 10 weeks, every 11 weeks, or every 12 weeks. In certain embodiments, boosting immunizations can continue until a protective anti-filarial parasite antibody titer is seen in the subject's serum. In certain embodiments, a subject is given one boost immunization, two boost immunizations, three boost immunizations, or four or more boost immunizations, as needed to obtain a protective antibody titer. In other embodiments, the adjuvant in the initial prime immunization and the adjuvant in the boost immunizations are different.

Further, in various formulations of the proteins and/or immunogenic compositions, suitable excipients, stabilizers, and the like may be added as are known by persons of ordinary skill in the art.

The disclosed proteins, immunogenic compositions, and methods may be used to prevent filarial parasite infection in a subject susceptible thereto such as, but not limited to, a human, or a domesticated animal.

### EXAMPLES

### Example 1. Transcriptome and Proteome of Onchocerca volvulus

Parasite and Serum Samples. Parasite materials used for RNAseq and proteomic analyses were collected at the research facility at the Tropical Research Station, Kumba, Cameroon, and in Ecuador and Guatemala. Written informed consent was obtained. In cases of illiteracy, the participant made a thumbprint and a literate witness signed. Institutional Review Board (IRB) approvals were obtained from the National Institutes of Health, the New York Blood Center and the Tropical Research Station, Kumba. The individuals who consented to participate in the study were born, or had resided for more than 10 years, in endemic areas, and we reconfirmed to have, or not, microfilaria in their skin snips as well as any other clinical symptoms of disease, such as dermatitis, nodules and/or ocular lesions. In order to identify the putatively immune individuals, biopsies of the mf-individuals were also tested for the presence of the 150-mer DNA repeats specific for O. volvulus using PCR and Southern blot. Samples were collected before the introduction of ivermectin or from subjects that had not received ivermectin treatment prior to the studies. Adult worm samples were obtained from nodules excised during nodulectomies. Briefly, individual and cleaned freshly obtained nodules were immersed in 0.5% collagenase (Sigma Aldrich, grade IV) in RPM 1-1640 containing 10% FCS supplemented with 200 units of penicillin and 200 μg/mL streptomycin. The flat tubes containing the nodule were then placed in a rocking water bath and incubated at 35° C. until the tissue was digested completely. Alternatively, frozen nodules were thawed, cleaned and digested with LIBERASE® TL (Roche) in Hanks Balanced Salt Solution (HBSS) supplemented with 3 mM CaCl₂. When digested, the liberated adult worms were unraveled from residual tissue with mounted needles under a dissecting scope, and then washed in several changes of RPM 1-1640 or HBSS. The cleaned adult worms were stored at −80° C. until use.

L3 larvae were produced at the Tropical Medicine Research Station, Kumba, Cameroon. They were obtained from Simulium damnosum flies 7-8 days after infection with skin microfilariae. After dissection and washing, the larvae were cryopreserved and shipped to the USA. Fresh L3 larvae were also cultured in vitro in groups of 10 larvae in 96-well plates containing a 1:1 mixture of Iscove's modified Dulbecco medium and NCTC-135, 20% FCS and antibiotic-antimycotic (Life Technologies, Gaithersburg, MD) for 3-days at 37° C. Larvae were collected after 1, 2 or 3 days in culture, washed with Tris-EDTA and snap frozen in liquid nitrogen.

Nodular and skin microfilariae were also purified. Embryonic stages were purified from mf and eggs that were extruded into the medium during the cleaning process. The medium was collected and centrifuged at 1000 rpm for 10 min at room temperature. The pellet containing the mix of microfilariae and embryonic stages was resuspended and layered on LSM (MP Biomedicals, CA) and centrifuged at 500 rpm for 15 min with the brake off. The purified embryonic stages that formed the pellet were washed and stored at −80° C. until use.

Transcriptome Sequencing, Assembly and Analyses. High-throughput transcriptome data were generated from the RNA of O. volvulus stages: nodular microfilariae (NodMF), skin microfilariae (SknMF), L2 (OvL2), L3 (OvL3), L3 day 1 (OvL3D1), L3 day 3 (OvL3D3), adult male (OvAM), and adult female worms (OvAF). For all larval stages and adult worms, RNA was prepared using TRizol® and lysing matrix D (1.4-mm ceramic spheres) and a FASTPREP24® (MP Biomedicals). RNA-seq libraries were prepared following the RNAseq protocols of the Illumina mRNA-Seq Sample Prep kit and the Illumina TRUSEQ® kit. Transcriptome libraries were sequenced on Illumina HiSeq 2000 machines. De novo assembly was done and is reproduced herewith slight modifications. Reads were trimmed of low quality regions (<13), and only those with an average quality of 20 or more were used. Illumina primers were removed from the sequences following a parallel BLASTN of the reads against HiSeq TrueSeq adapters. Resulting reads were assembled with the ABySS software (Genome Sciences Centre) using various kmer (k) values (every fifth from 21 to 91). Because the ABySS assembler tends to miss highly expressed transcripts, the SOAP denovo-Trans assembler was also used, again with odd kmers from 21-91. The resulting assemblies were joined by an iterative BLAST and cap3 assembler. Coding sequences (CDS) were extracted using an automated pipeline based on similarities to known proteins or by obtaining CDS containing a signal peptide. CDS and their protein sequences were mapped into a hyperlinked Excel spreadsheet. Signal peptide, transmembrane domains, furin cleavage sites, and mucin-type glycosylation were determined with software from the Center for Biological Sequence Analysis (Technical University of Denmark). Reads were mapped into the contigs using BLASTN with a word size of 25, masking homonucleotide decamers and allowing mapping up to three different CDS if the BLAST results had the same score values. Genes that had blast scores<30% of max possible score (self blast) in other nematodes with an e-value greater than 1E-05 were considered as ‘unique’. To be O. volvulus unique, the genes were compared with the genomes of O. flexuosa and O. ochengi. Automated annotation of proteins was based on a vocabulary of nearly 290 words found in matches to various databases, including Swissprot, Gene Ontology, KOG, Pfam, and SMART, Refseq-invertebrates and a subset of the GenBank sequences containing nematode protein sequences, as well as the presence or not of signal peptides and transmembrane domains. Protein repeats were analyzed using repseq and reptile (www.reptile.unibe.ch) algorithms. Further manual annotation was done as required.

Transcriptome data (RPKM) from Excel spreadsheets was imported into JMP Genomics (SAS, Inc.) for general assessment of distribution analyses, correlations, principal component analyses, analysis of variation (ANOVA), hierarchical clustering, and heatmap generation, parallel co-ordinate plots. Heatmaps of clustering analyses were also done in R using array of packages. Differential expressing of genes was analysed using DESeq. Two replicate samples Ov1F (male) and Ov4F (female), were observed to not be exclusively male or female (pre-analyses) and were excluded from all stage-specific analyses. However, they were used for differential expression analyses with the rationale that any contaminating female transcripts present in the male sample would result in the differentially expressed genes with lower adjusted p-values to drop off and thus enriching for highly expressed genes. Likewise, any male transcripts in the female (including contributions from stored sperm and embryos) would lead to drop-off of lower range of genes and selecting for the most highly regulated genes.

Protein Depletion, Denaturation, Digestion, and Desalting. For proteomic analyses, additional stages of embryos (OvEMB), L3D2 (OvL3D2), and L4 larvae (OvL4) were also analyzed. Total soluble proteins from all the stages were extracted using the UPX universal protein extraction kit (Protein Discovery) as per manufacturer's instructions and quantified using PIERCE® BCA assay (ThermoFisher Scientific). Extracted protein samples were prepared for digestion using the filter-assisted sample preparation (FASP) method. Briefly, the samples were suspended in 1% SDC, 50 mM Tris-HCl, pH 7.6, 3 mM DTT, sonicated briefly, and incubated in a Thermo-Mixer at 40° C., 1000 RPM for 20 min. Samples were centrifuged to clarify and the supernatant was transferred to a 30 kD MWCO device (Millipore) and centrifuged at 13000×g for 30 min. The remaining sample was buffer exchanged with 1% SDC, 100 mM Tris-HCl, pH 7.6, then alkylated with 15 mM iodoacetamide. The SDC concentration was then reduced to 0.1%. Samples were digested overnight using trypsin at an enzyme:substrate ratio of 1:100 at 37° C. in a Thermo-Mixer at 1000 RPM. Digested peptides were collected by centrifugation. Twenty micrograms of the digested peptides were desalted using reversed phase stop-and-go extraction (STAGE) tips. Peptides were eluted with 80% acetonitrile, 0.2% trifluoroacetic acid and lyophilized in a SPEEDVAC® (ThermoFisher) to near dryness, approximately 1 hr.

Protein Array Construction. The following cDNA libraries from OvAM (SAW98MLW-OvAM), OvAF (SAW98MLW-OvAF), OvL2 (SAW98MLW-OvL2), OvL3 (SAW94WL-OvL3), molting L3 (SL96MLW-OvML3), and MF (SAW98MLW-OvMf) were obtained from the NIH/NIAID Filariasis Research Reagent Resource Center (www.filariasiscenter.org) and used to amplify selected gene products. Molting larvae transcripts that were not amplified successfully from the cDNA libraries were subsequently obtained from oligodT cDNA prepared from RNA purified from OvL3D1, OvL3D2 or OvL3D3 (SUPERSCRIPT® III First-Strand Synthesis System, Invitrogen). In vivo recombination cloning was performed. Briefly, PCR primers were designed as 40 mer oligonucleotides with 20 sequence specific bases and a 20-base adapter sequence. The adapter sequences were designed to be homologous to the cloning site of the linearized T7 expression vector pXT7 and allow the PCR products to be cloned by homologous recombination in E. coli DH5a cells. PCR reactions were set up using HOT MASTER MIX® (5 Prime) plus DMSO (5%). The recommended cycling conditions were used and PCR products were checked for correct size using an agarose gel. PCR products were mixed with linearized pXT7 vector and were transformed into DH5a competent cells. DNA was purified using QIAPREP 96Turbo® Miniprep Kit (Qiagen). Resulting clones were checked for insert on an agarose gel and were sent for sequencing (Retrogen).

Chip Fabrication. Proteins were expressed using a coupled in vitro transcription and translation (IVTT) system, E. coli based cell-free Rapid Translation System (RTS) 100 High Yield Kit (5 Prime), from the O. volvulus expressible clone library following the manufacturer's instructions with the exception of adding detergent to the IVTT master mix at a final concentration of 0.1% Brij 78. Shortlisted O. volvulus proteins were synthesized using IVTT in disulfide-bond folded formats and printed onto an array. Known immunogenic proteins (purified recombinant proteins) were also printed as positive controls.

Approximately 1 nL of unpurified IVTT reactions were spotted onto 8-pad nitrocellulose coated ONCYTE® Avid Slides (GraceBio Labs) using an OmniGrid Accent microarray printer (Digilab) equipped with a Avid™ 946 Printhead and 946MP4 Spotting Pins (ArrayIt). Each IVTT expressed protein includes an N-terminal 10× polyhistidine (HIS) epitope tag and C-terminal hemagglutinin (HA) epitope tag. Microarray chip printing and protein expression were quality checked by probing random slides with mouse anti-polyHIS (Sigma), rat anti-HA (Roche) and rabbit anti-E. coli (LifeSpan BioSciences). Antibodies were diluted 1:1,000 in a 3 mg/mL E. coli DH5a lysate solution in protein arraying buffer (GVS Filter Technology) and incubated at room temperature for 30 min. Chips, FAST® Slide Holders (GVS Filter Technology) and FAST® Slide Incubation Chambers (GVS Filter Technology) were assembled and nitrocellulose pads were hydrated using 100 μL blocking buffer for 30 min at room temperature with rocking. Blocking buffer was removed, pre-incubated antibodies were added and chips were incubated for 2 hr at room temperature, washed three times with 1×TBS-0.05% TWEEN® 20, followed by incubation with Cy5-conjugated goat anti-mouse IgG Fcγ, Cy5-conjugated goat anti-rat IgG Fcγ or Cy5-conjugated goat anti-rabbit IgG Fcγ (Jackson ImmunoResearch) diluted 1:400 in blocking buffer for 1 hr at room temperature with agitation. Chips were washed three times with 1×TBS-0.05% Tween 20, three times with 1×TBS, and once with water. Chips were air dried by centrifugation at 500×g for 10 min, stored in a light proof desiccator for at least 2 hr and scanned on a GENEPIX® 4300 with Autoloader (Molecular Devices) using the 635 nm laser. Resulting 16-bit TIFF images were quantified using GENEPIX® Pro Microarray Analysis Software (Molecular Devices) and a GENEPIX® Array List (GAL) file. Spot and background intensities were measured and median spot values minus local background (M635-B) values were exported as comma delimited file (CSV).

Probing Samples. Serum samples were diluted 1:100 for IgG and 1:50 for IgE in a 3 mg/mL E. coli DH5a lysate solution in protein arraying buffer and incubated at room temperature for 30 min. Chips, FAST® Slide Holders and FAST® Slide Incubation Chambers were assembled and nitrocellulose pads were hydrated using 250 μL blocking buffer for 30 min at room temperature with rocking. Blocking buffer was removed, pre-incubated serum samples were added and chips were incubated overnight at 4° C. with agitation. The following day, chips were washed three times with 1×TBS-0.05% TWEEN® 20, followed by incubation with biotin-conjugated anti-human secondary antibodies against IgG1, IgG3, IgG4 or IgE (SigmaAldrich) diluted (1:1,000 for IgG, 1:500 for IgE) in blocking buffer for 1 hr at room temperature with agitation for one hour. Chips were washed three times with 1×TBS-0.05% TWEEN® 20, followed by incubation with streptavidin-conjugated SURELIGHT™ P-3 (Columbia Biosciences) at room temperature protected from light with agitation. Chips were washed three times with 1×TBS-0.05% TWEEN 20, three times with 1×TBS, and once with water. Chips were air dried by centrifugation at 500×g for 10 min, stored in a light proof desiccator for at least 2 hr and scanned on a GENEPIX® 4300 with Autoloader using the 635 nm laser. Resulting 16-bitTIFF images were quantified using Innopsys Mapix Software and a GAL file. M635-B values were exported for each slide as GPR files.

Data Analysis. Software developed in R (Antigen Discovery Inc) was used to process the individual GPR files in batch to create a single matrix of the raw data and to perform automated data quality checks. The raw data were normalized by dividing the IVTT protein spot intensity by the sample specific median of the IVTT control spots printed throughout the chip, then taking the base-2 logarithm of the ratio. The normalized data provides a relative measure of the specific antibody binding to the non-specific antibody binding to the IVTT controls. Normalized data was imported into JMP Genomics (SAS) and analyzed for antigen reactivity and significance (ANOVA) between the clinical groups and isotypes, and adjusted for multiple comparisons. Significant proteins were graphed in Prism 6.0 (GraphPad).

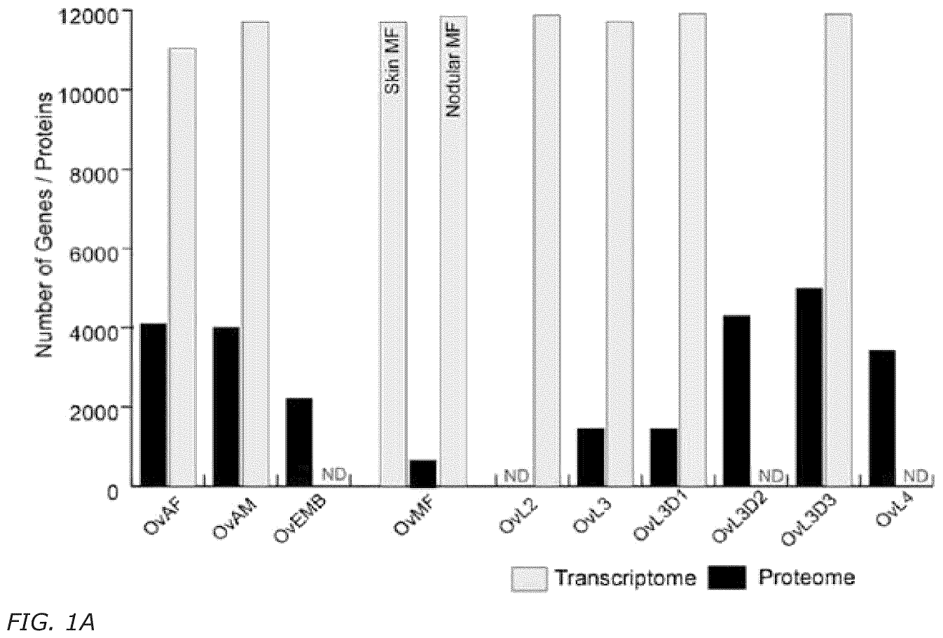

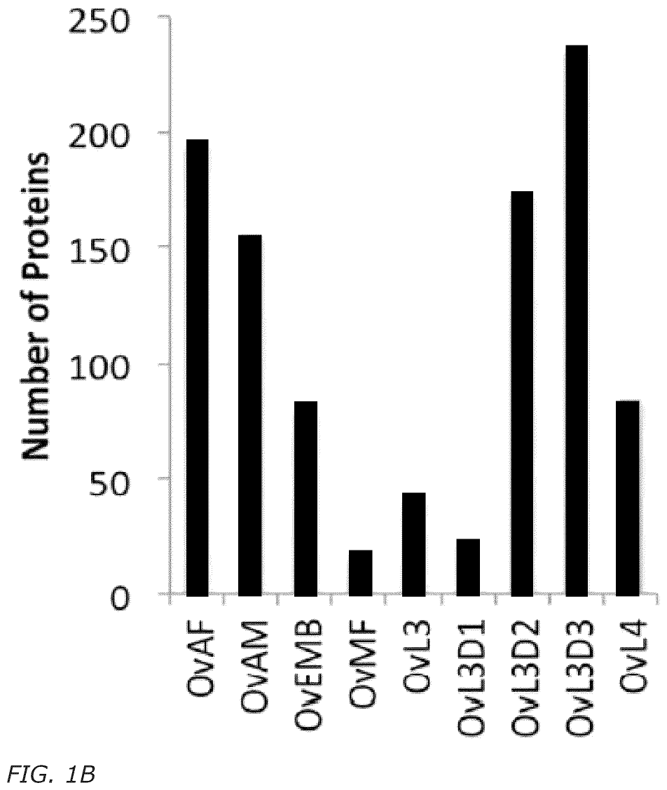

Transcriptional profiling by RNAseq resulted in the identification of transcripts corresponding to 99% of the predicted genes (FIG. 1A) across all the stages in the parasite lifecycle. Over 75% of the genes had 100% transcript coverage in all the stages except the adult female which may have been related to the age/condition of the worm(s) inside the nodule, degradation of RNA during the digestion of the nodules, or the fact that the majority of the adult female worm is comprised of uterine tissue and embryos. Several transcripts with less than 1 RPKM were subsequently identified and verified by mass spectrometry (in proteomic analyses) and thus have not been excluded. Shotgun proteomics identified proteins with a median coverage range of −10-15% from each of the stages profiled. A total of 7,774 O. volvulus proteins were identified across all the stages (FIG. 1A) resulting in the validation of over 64% of all predicted proteins. Though there were no differences in the number of transcripts identified in each of the stages, maximal proteomic coverage was observed during the L3 to L4 development and in the adult male and female worms (FIG. 1A). This approach also resulted in the identification of 465 of the 785 putative Wolbachia proteins (FIG. 1B). Wolbachia is a genus of bacteria which infects some nematodes. Wolbachia species have been found to be endosymbionts of O. volvulus adults and microfilariae, and are thought to be the driving force behind most of O. volvulus morbidity. Overall r-values for correlations across all of the stages between the transcriptome's RPKMs and proteome abundance ranged from 0.25-0.39, values that are considered acceptable for global comparisons.

Multivariate analysis revealed stage specific transcript profiles that segregated the vector-derived stages (OvL2, OvL3), the early human developmental stages performed in vitro (L3 to L4 molting: L3D1, L3D3), the adult male (OvAM), the adult female (OvAF) and the microfilarial stages (SknMF—mf obtained from skin, NodMF—mf obtained from nodules). Infected nodules often contain more male worms than female worms which has been attributed to adult male migration between nodules. The proteomic analyses indicate a probable bias towards a male-like expression profile as the worms develop from L3 to L4 and to young adults. Hence it was hypothesized that it is also likely that proportionately more male worms develop from a single infection. Indeed, structural gender differentiation can be observed in in vitro developing L4 larvae. Notable among transcriptional and proteomic profiles was the observation that, compared to all other stages, the adult males have higher transcript abundance levels with many differentially expressed genes.

### Example 2. Stage-Specific Functional Enrichment

Functionally, the total putative proteome was classified into functional categories. Forty-four percent of O. volvulus genes have no yet known function. The distinctive biology of O. volvulus is likely to be underpinned by genes with potentially novel functions and with relatively few homologues in other helminth parasites. Approximately 9% (1173) of the predicted genes in the O. volvulus genome encode unique genes with less than 30% homology with other nematodes. 92% of these ‘unique’ genes are hypothetical or genes of unknown function of which 7% are potentially secreted. Clustering of these unique and divergent genes based on transcript and protein abundances indicates distinct subsets that are enriched in specific stages, and that these clusters have signatures of being able to be secreted (“secreted-divergent”). Although largely uncharacterized, the stage specificity of their expression is an indication of their developmental regulation and may allow for functional assignments in the future.

Gene Set Enrichment Analysis (GSEA) demonstrated that the female stages were associated with pathways linked to detoxification and the extracellular matrix. This enriched subset of extracellular matrix related genes was primarily comprised of collagens and chitin. Although the microfilariae are an integral part of the fertile adult female, genes corresponding to NADH dehydrogenase activity (GO:0008137) and cytochrome-c oxidase activity (GO:0004129) were highly represented in adult females. In contrast, the microfilarial stages showed significant enrichment for processes associated with protein synthesis (ribosomal proteins) and protein modification with cyclophilins and chaperones (heat shock proteins) being the major contributors. These are likely the machinery required for cellular morphogenesis that occurs after being ingested by the blackfly vector.

### Example 3. Secretome and Host-Parasite Interactions

The O. volvulus genome encodes ˜20% of genes predicted to be secreted by classical secretion and about −42% through non-classical secretion. All filarial helminths are known to release excretory/secretory (E/S) products that are critical components in the helminth arsenal of proteins that perform diverse functions that include: 1) modulating the host immune response; 2) host tissue remodeling; 3) alteration in host tissue nutritional status; or 4) enhancement of larval tissue migration. The O. volvulus genome encodes many of these immunologically relevant genes. Among the examples of the stage-specific enrichment of these immune-related gene products are the L3-enriched or mf-enriched cystatins and serpins that have been shown to interfere with antigen processing and presentation to T cells; the OvAM-enriched expression of indoleamine 2,3 dioxygenase (IDO); and the developmentally regulated L3/L4-enriched homolog of suppressor of cytokine signaling 7 (SOCS7; OVOC678). Proteases (serine, aspartic, cysteine and metallo-) are integral to host invasion, developmental molts and migration in a number of nematodes. Serine protease inhibitors also play an important role in controlling the molting process and immune evasion. The analysis of the Ov genome revealed the presence of 18 serine protease inhibitors, nine of which are highly expressed during the L3 to L4 molt. Four of these are SPI-like, probably having resulted from a duplication event of Ov-SPI-1 and Ov-SPI-2; their marked expression during the L3 to L4 molt is consistent with not only their role in early larval development but also in their putative role in immune evasion during their early adaptation to their human host. Interestingly, two of the serpins are also highly expressed in adult males indicating a potential role in spermatogenesis, while one is highly expressed in both nodular and skin mf.

### Example 4. Immunomics

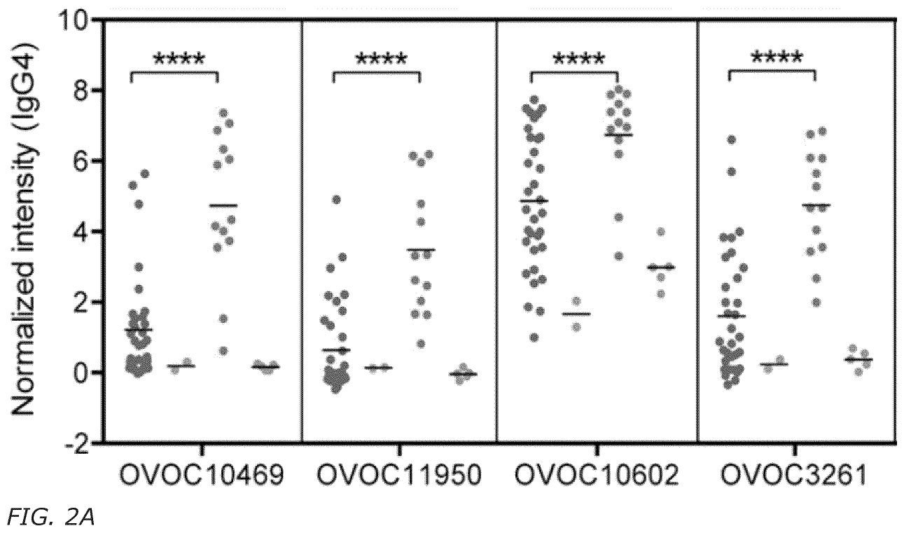

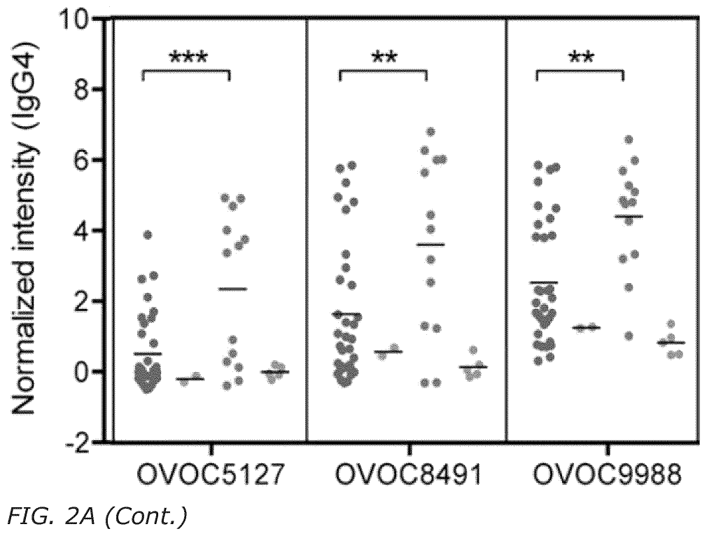

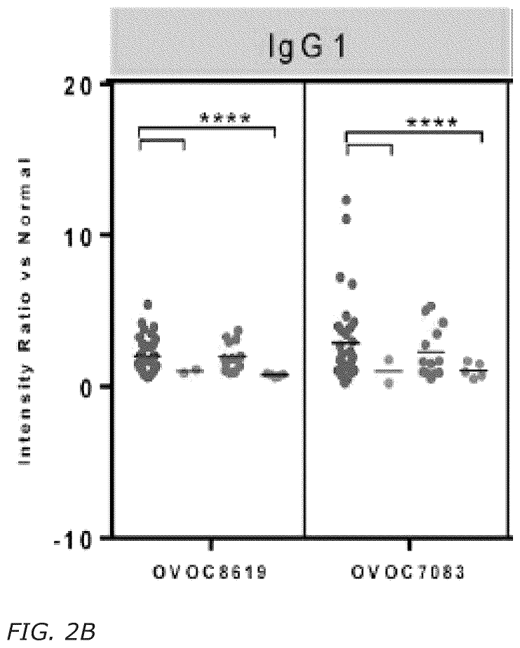

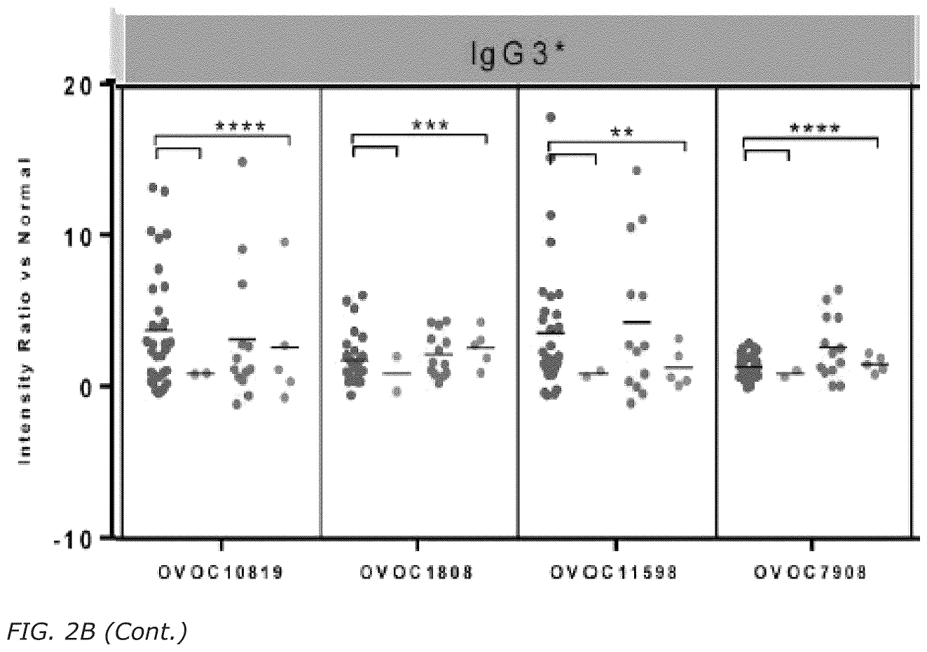

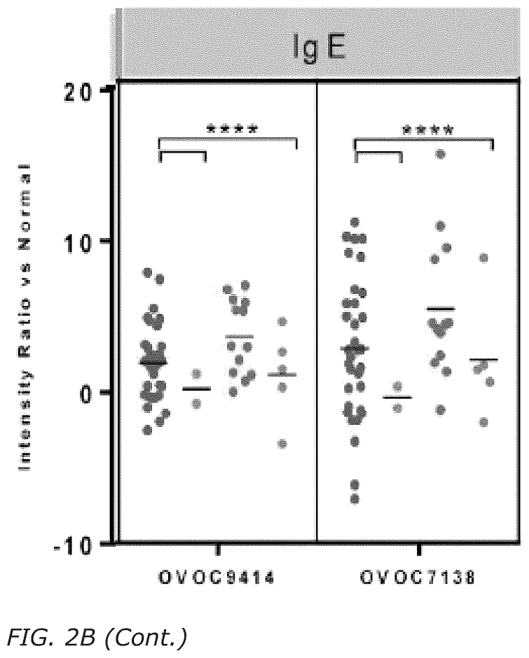

Using an immunomics approach, host antibody responses to candidate parasite antigens were profiled. Selected (397) proteins (based on their elevated expression in infective stage larvae and during molting and/or in microfilariae and/or adult stages) were printed as protein microarrays, quality checked, and assessed for isotype-specific responses (IgG1, IgG3, IgG4, IgE) with 52 individual sera comprising O. volvulus-infected, putatively immune, and control individuals from Ecuador, Guatemala, and Cameroon. After normalization, clusters specific for IgG4, IgG3 and/or IgG1, and with or without IgE reactivity were identified. Heretofore unrecognized biomarkers of active infection were identified (e.g. OVOC10469, OVOC10602, OVOC11950, OVOC3261, OVOC5127, OVOC8491, OVOC9988) as seen in FIG. 2A and Table 3. Further analyses led to the identification of potential novel vaccine candidates (e.g. OVOC10819, OVOC5395, OVOC11598, OVOC12235, OVOC8619, OVOC7083) (FIG. 2B and Table 4), based on IgG1 and/or IgG3 reactivity (with little to no IgE reactivity).

TABLE 3
O. volvulus biomarker sequences

| SEQ | Protein | |
|---|---|---|
| ID NO. | Description | Sequence |
| 1 | OVOC10469 | NIAFAPNPKDSNNELFADAESALGSEYAQFVEQSKQHKPVYFSDNQNTLETIKLE |
| | Secreted protein | SIPNPETETAYPMFICGFLGCMKKMNSVEEYLEHFKMHEKQGY |
| 2 | OVOC11950 | YPTEKETVEPIDTMVKDDIDLVKAEVAEAEEADVEKEVAELTEEEAAEIAEVL |
| | Secreted protein | DEMEEEFFAFLLFDFILDLFRETLEKNSESQEASIDEVMPEIQGVSAEEA |
| 3 | OVOC10602 | FRTQSIGIRGRLMCGSKPASNERIKLWEEDSDDLLDQGYTDENGEFLLKGDTVEL |
| | Conserved | TPIDPVFKVYHDCDDGIKPGKRKVKFKIPKSYITEGKTPKKIFDLGTLNLETIFN |
| | secreted protein | DEERELIVT |
| 4 | OVOC3261 | SYCEDWDPEDFPSFVLKLSQNATEEFCELYEMEMEVPINKFYDMLRKWAEKYSVQ |
| | Secreted protein | AETNRFIAEEMNYDKMQSKVLMERLQASNGTTEVKGVLEKALKLQESMHLSPDYI |
| | | QNVIDTMMENLPIDKQNEATLLWNSLYPDDIYNECGPRF |
| 5 | OVOC5127 | APPNRDTADDLQNADMQRQWEQEQRQREEVQKEEIAKYVKYMRYLETVLNILQAT |
| | DNA binding | PQWKEAMQSMTQEEMRGGKIAEMVDKLEPHIIEQLAKAKILELQRLEQEIKDQLN |
| | protein | ADGGATHNIKVSEILTVCLKEGFKKPSNLGIPEHLDFNNWETFSQEDLRKLIVKI |
| | | VTDMDELEEQRKQDFKQYEMKKKAEEDHKMQAKMIQTEREEYIRQMEEQRRRHNK |
| | | HEPLKHPGSRNQLRKVWEDTDKLDKDAYDPTTLFGLHDRNNDGYWSYDELNTIFL |
| | | PEIEKLNNFSDVERLEELYRMRDHVMKQMDTDGDHRISRAEFLADREAQEEKPDQ |
| | | GWEDIGDKDQYTKEELEIFEKEYAKQQGWGEYAYSTPAPTPDPSRMIQPDQAPMQ |
| | | RLDAPSDQVGDMFAQQSHQIPVKHVEPIQSVQQQQMDEVNS |
| 6 | OVOC8491 | FPTEANPAVGTDNAHEDNLTTEEKMQLKKFAKTNAANFSLTDPEFIDGLKNEAAG |
| | Fatty acid | LFSKLTGLRDIINAKLDTMQPESRLFIEKLLRRFLAAFSHDGLMNILESLKGFGK |
| | retinoid | EVIDMFDGLSRPIQNDILNAFPLVGSYITSDIARLMLRKLAELDLLSRKSTLTPT |
| | binding protein 2 | VDQFNDDSGKHFPRPQVIEPEEPENSDPEDAQSTDYGKKKVVTTTTFPIITGEED |
| | | EILVKKIVENK |
| 7 | OVOC6759 | IPLPEELDYDGEIPNCRDGEKPLLAADIGVYTCDKNCPKGFRCEYRTMDSTSKKG |
| | Conserved | ICCPNLKELAKIYSEDEEVDKSIKKSNI |
| | secreted protein | |
| 8 | OVOC451 | MISCFALPFPHVCYMAYCTQVIASIMKGWNQNFRFSTVIYLFRNIFSSSVISCVN |
| | Filarial antigen | MILSSTFYALLFVSAVVIVEAMPASESTYSVIIIRINDTTCKIEDGVVSVNGQVI |
| | Av33 | GNLTEEQKEELEAYNVQTQGWFQQLHQKIEELFKTFFGSIKSMWKHSPISGSESS |
| | | PQSSTPDNIITDKLDDQDRRLKDQGDSENSSLFGLKLPSFCKVN |
| 9 | OVOC12329 | FRSLKIGRKQSTAVKGVLTCNGKPAVNVKVKLYNDSQGRYVENSMDEGKTDSEGR |
| | Conserved | FLLQGHETSITSIDPILKLYHNCDVENAQCLKRFSILIPNDFVSEGLEPKKTFDM |
| | secreted protein | GTLNLGGKFFDEGRECAS |
| 10 | OVOC3337 | QIIGSFNGNYAGDGSLNNNANSFGERTTTTRSTSRPSLPPRPGYPSRPGYPFKPG |
| | Glycine-rich | FPPRGPPIPYPHGKPSGPRYPCYGGYGGYGHPGYGPFGGNGYLGYTVCSGRGEFG |
| | cell wall | GYGPGLGGGTGLGGLGPGEFGGYGPGLGGGTGLGGLGPGGFGGIGPGLGGGGGLG |
| | structural | GPGRGGFAGYGPGLGGGRGLGGPGPGGFDGYGPGLGGRPYPGGYGRFYGPGPYPG |
| | protein-like | DRLDPRGLSESGRPRTRLASYNRNDRGTQFSYIRDR |
| 11 | OVOC10264 | MTNEYETNYPVPYRSKLTESFEPGQTLLVKGKTAEDSVRFTINLHNTSADFSGND |
| | Beta- | VPLHISVRFDEGKIVFNTFSKGEWGKEERKSNPYKKGDDIDIRIRAHDSKYTIYV |
| | galactoside- | DQKEVKEYEHRVPLSSVTHFSIDGDVLVTYIHWGGKYYPVPYESGLSGEGLVPGK |
| | binding lectin | SLLIFATPEKKGKRFHINLLKKNGDIALHFNPRFDEKAIVRNSLIAGEWGNEERE |
| | | GKMILEKGIGFDLEIKNEEYAFQIFINGERYATYAHRLDPREINGLQIGGDLEVS |
| | | GIQMR |
| 12 | OVOC4230 | DLLSEAGDFFTKHFTDIKSLFAKDEKQLQQSVDRVKDLLATIQDKMSMLQPLAND |
| | Conserved | MQKTTLGKIGDLISQVNSFRETMSNPKMDFTNKENKWEELLKKIFVTEGLNKVIP |
| | secreted protein | LLQKLKNSAPTTFATYLFTCIVPVLINTLRE |
| 13 | OVOC10384 | MARINRLNFLLCIVHANITSAPNPKDSNDELFADAESALGSEYAQFVEQSKQHKP |
| | | VYFSDNQNTLETIKLESIPNPETETAYPMFICGFLGCMKKMNSVEEYLEHFKMHE |
| | | KQGY |
| 14 | OVOC8422 | FSWKFGERLDEPVLMLRDLRAKEISPPSYMKRFESDTNEQLLRYILHPKMLRRHD |
| | secreted protein | LSNALFYQPLWKMR |
| 15 | OVOC6395 | MEGSPIKETRGLEATPVFEMVRSATLTFLLAVSTVLVVSRPNVLLPPKLPWDSDW |
| | Protein | RQKPPPFPPEPPPEFKGILPPEIFAKLTAIHQDQSLTIPQKIVKIEEIMNSLPED |
| | LOAG_00657 | VLQRLPLPPVFRLLPQNVQEMIKTVRTTKNLTMEEKWLQMIILIESLPKQQHRLL |
| | | QQMLPKFSLGPLPDFQDIIPKEDWDKLTAVYQDTNLDNIEKLRRVDEIIDALPDS |
| | | IRQKIPLSPPFQKLPDHIQQQLQIIHTERGLTTEQRFRKMKAIIESLPWDMKKLM |
| | | FQP |
| 51 | OVOC10469_Pep2 | EQSKQHKPVYFSDNQNTLETIKLESIPNPETETA |
| 52 | OVOC3261_Pep1 | CPSLSSYCEDWDPEDFPSFV |
| 53 | OVOC3261_Pep3 | LPIDKQNEATLLWNSLYPDDIYNECGPRF |
| 54 | OVOC10469_Pep1 | AFAPNPKDSNNELFADAESALGSEY |
| 55 | OVOC10469_Pep3 | GCMKKMNSVEEYLEHFKMHEKQGY |
| 56 | OVOC3261_Pep2 | INKFYDMLRKWAEKYSVQAETNRFIAEEMNYDKMQS |
| 57 | OVOC5127_Pep1 | APPNRDTADDLQNADMQRQWEQEQRQREEVQKEEI |
| 58 | OVOC5127_Pep2 | TDMDELEEQRKQDFKQYEMKKKAEEDHKMQAIQTEREEYIRQMEEQRRRHNKHEP |
| | | LKHPGSRNQLR |
| 59 | OVOC5127_Pep4 | DREAQEEKPDQGWEDIGDKDQYTKEELE |
| 60 | OVOC5127_Pep5 | TPAPTPDPSRMIQPDQAPMQRLDAPSDQVG |
| 61 | OVOC5127_PepX | VSEILTVCLKEGFKKPSNLGI |

TABLE 4
Immunogenic composition O. volvulus protein sequences

| SEQ | Protein | |
|---|---|---|
| ID NO. | Description | Sequence |
| 16 | OVOC8619 | LIKVFPEISANMSVMFANSRSNQANNGYLVEFKAGRSNLQAGSTVDRRKV |
| | Adhesion- | VADKTKGLVFIKQSSDQLMHFCWKNRETGAVVDDLIIFPGDTEFLRVRECT |
| | regulating | DGRVYMLKFKSTDEKRLFWMQDGKTDKDDENCKKVNETLNNPPAPRAAA |
| | molecule | RGGADRADVSSFGTLAALGSAGAESELGALGNLDQSQLMQLLSLMNHTN |
| | | STSASEATNLLPQLPLVADTSHPMTSEDSGTTSTHGATPSNTPANGIVADS |
| | | SSNNAMQLSQLKEIIASITPPDGSGRKPSIDFTDVLCCADKINDVLRKYAEQL |
| | | IPHLPSQEPIYNNQEELQQTLRTPQFRQAADIFGHALQTGQLAPVLRQFGID |
| | | GNTATAAGNGDMVAWAAQFTTAENGKEITAKTETSPSQPGMESDVEDEE |
| | | TNEKAIRETEKNRTDDHMDLD |
| 17 | OVOC7083 | MNYKAPIELQQLLSITKMLSLSVLLLFTSMAIMARPPNSDEIKELRQQQLNES |
| | Secreted | KDDYDTLPDVNHIPESFKESLKKQKMLYLDMLRQHNL |
| | protein | |
| 18 | OVOC4111 | LSVPAGLRPAKKVGDPKEQIVPGKQQQLQQQQQQQLLQQQQQQQQQQQ |
| | Mediator of | QQQQQQQQQQQQQQQQQQQEQQEELQQLQESEETGEEHRQQQQQQH |
| | RNA | DEALTLSPTPKVPPNLSIRSRMMAALSASVGESNKEKNSSNDETDNSSKST |
| | polymerase II | NSPSKPPIIFPKANKKTVVGKIAPSGISKGSARVIVAPPSKLGTNNFGLNTVL |
| | transcription | QTNLVDSRGRIMKNVNSVPIKVPSSAEMRNARTRHTARQVESDADKVVPIK |
| | subunit 15 | FGSTSRRR |
| 19 | OVOC1808 | NNSNLDISMREKNAVNAIEKQDLPRSHRFKRQYSCGQCGGGGGPPVVVS |
| | | PCQQCKGGGAGVSAIGGAGGISAIGGGVSAIGGGFGGGGGDTVAVVCCG |
| | | ATGLKGMFRNWWLHIPLLLLPMSMSWIKALFL |
| 20 | OVOC11598 | YYVPDNYWPLRIIGYHHIPVMINMWYLFQTEISNIGVDAVLVQSPLYRTLTP |
| | Secreted | DVVHDIISINVEPNHTVVVEQSNPMLQASSVEQAPAAAPLSITLIAPGITISRT |
| | protein | HKVDTYKSTMEMYDADKLHSNEIFKRRVRKMVLPPSRGEEVRKPPSSTDG |
| | | YESENVESYGQKGVEQAPPEIEQYVKKKK |
| 21 | OVOC3901 | MKYCLSSIIAATIATTTTTATAIIATTITAATISVAPFHASSPSSSLSSSSFSSFF |
| | Immuno- | LVLPLITTILLIVPEQAHSTATVTEHRSPPDLSIPSQTEFRVPVGTKQFRLICP |
| | globulin I-set | VKEKNDDLLMIQWKKNDEPIGFDFNNRFKLARSDRELKIRNPQLSDGGIYQ |
| | domain | CQVVNGFGHRELNFTVTFYDPAMENDQNTDSTLTLTTKASPPIWKNETEIR |
| | containing | NWMINPVRITIGGALLLKCPAKGNPLPHITWLRDGKVLEREITYHYSSAILYL |
| | protein | SDVQPSEGGKYICKLENEHGSIEASFHVYVENFFEGLDGESWSIDQTNAQL |
| | | YPVIDEPFNNTVRVGRTAQFQCKVKNQQQPLIKWLKRVEDPNAIRQTNAN |
| | | ATLIHANNMHLLLLEKPETSAELSDGISLNRLIIPNVRYEHSGTYLCVVTNAR |
| | | GDIAYRSAYLNVIARSDHGELSNLYFYGGLLVLIVVFTLITYAVHFLRKNQAA |
| | | KSTESAPGITNIRYSFSLRPPPPNLPPPKAPALPSERQQLMPNNQPCDRYT |
| | | VNSAAATYYPQFATPDKKLQKIITESGTRPTPIRRTNGGDTKYRLKDDYISS |
| | | PKWVHAKGDNIEVEMDQNLLKNRSTHCHNPVSIAYGRIDNIDRQQQKSFLT |
| | | IGNLQKR |
| 22 | OVOC10819 | KEIIWDCYGDYEECVAESSKMDHVDVNNVESRNIIEFCSDHTQNILPCLATK |
| | Secreted | LGLIKSMSVSMFSLLLTICEAETRNNRPAATEVQQILKHLARLYAYFCAYSN |
| | protein | VIDLRYNKECFRYLKKRCILNKPDDSCIFHHCGEKNLNLSESSPFIQQHKTTI |
| | | INQLNQSATFKNYHHRITTIFTVIITFISMIQ |
| 23 | OVOC5395 | MYNQENHDKRRNDDRFILSLPFGTNVENKSYFKPIKLSNPYSDKYLEVNKK |
| | Protein | SSDDSDQNLNQALSVPQSNYDQSSESLSIDDSDLIDDSTSAAQLSTSSPISV |
| | Bm1_06245 | TSASTSSFYPTLNIGNGMEISAKYAKLEQSQGIKSDQSTSRVSDRYKKYTA |
| | | VKRRLSELYGIIEEKDEQLRVVRNELNGKDLEIGKLCDKIRALEYNCGRLQS |
| | | MIESAGDESDQNQVKLHEIINERDGLLIRNASLSRQIEFEKREWSIERERLS |
| | | MDLDDVTRELELQKMILNGESISEIVQRWQTKVFELEGMITDRDRAIRAQQ |
| | | VQISKLKESIAETDRISCADSSESQTKFDFPSFTYIKRLLLQYLTRLADLHFSS |
| | | DEERMQLVRNMSSILHLSDEEQRQVWANLKSKIQIS |
| 24 | OVOC12235 | QCPTGSVSLLSGYRCTSSIQCQTIIPGSYCYYGVCCTGGSDVLSKTVSYGG |
| | Conserved | YCTMTVQCSTTGATCISNICQCDINSHYNGHSCVSISNFCPSNQVFIKGECY |
| | secreted | RKVTYGFLCNYTQQCGYIGAFCIGNICSCQLDYTFDGSKCIPRSRICPANQI |
| | protein | AIGGQCYPSARFGERCLYSEQCIDRWYRSLSCVNGFCNIRNDDDISKPKC |
| | | RNPRAEVEYVNGTAKNCLYWPCTVGYFCEYAGGMNGGRYICCGTNANKI |
| | | YGKVQLYPGTGTPLQCTEIGRCPFPDTPNCVMSYRYGYKVCCSTLN C |
| 25 | OVOC7908 | QETSEQPGLTVEIIAEQQDATTADQEVTTTVDTHHQHQHQTDKVVKSRQIT |
| | Lateral | GDEQTTTTTTAINLNETITNSTTDSNSTIITTTLDLQESTTTGTTDNHHHHHH |
| | signaling | HHHHHE |
| | target protein | |
| | 2 homolog | |
| 26 | OVOC7430 | MKQTTAWGNALCVLCNCHQPQIICPPPPPAVCPRVVCPPPRPPVCPPIYC |
| | RhoA GTPase | PPPVVCPPPPVCPPVPFCHSQICPPCGTHTVPVAVVGCCKGCACSVRFKR |
| | effector DIA/ | DSSSVNGLMLKKNLLCNNDQLMTIMEKKIGTNATEAAFAIKKEADSELKAKF |
| | Diaphanous | SVFCAMNDLIYVAHAESFCQHKKGDIICFAYKS |
| 27 | OVOC8936 | MDCKLILPFYILLANLEANAFHLSGYRSRSYLQGIQPYDIQPLDVQPQFIRVQ |
| | Microfilarial | TLKSQDIQPYSIQSRSEDQPCEGCKITISCGSKNCKSKKLPYVYKPIFKLLST |
| | sheath protein | RSTKKPVFTLPTQPPAQWDCPCPCHVPQRCRMCSACHESYI |
| 28 | OVOC5806 | NRIISRRLSLFIQQYCCNNISQIYRLNDCKYSKVKMEIDKKIFIIVSKTEWCNE |
| | Conserved | AIKVVFGKSAEAIRNNSDAISWLASYNYTGSMDLRSKWPYDAYFDNVTRTA |
| | plasma | HGLARIDLLCHKKRPQLGPRIWKRSVQKIKQKKDRPFAVNTYGNNKGLFTIT |
| | membrane | VGVLLYAAFGTCFLIANLAYLFGIYIIYDASIIDEVS |
| | protein | |
| 29 | OVOC4665 | IGENPMDVNAIAGIIGGISNMMQNNVETIDVPSSQIMGRWYQVYKAAIAFDV |
| | Conserved | YRTDIFCPVAYFKPNSVMGEDGFSIEEAYRVITKNGPVETYKRDLNKVGTG |
| | secreted | QYWMYTEEYFYPRQFNIISVGPNYTNTTDGSEEEKQYQYMVVTDGNRLSL |
| | protein | SVYARHPMIFYQKYNEEVVKFLEHAGFGGKVFWNSPKPIYQGADCEWPSE |
| | | KEVFARRVLKNQELAKNGGLDTATKSGSFGGSSQATDVRSSITEILQNPQL |
| | | ALQKLVQGH |
| 30 | OVOC8227 | MTIIKSMLKITHVIFDLDGLLIDTEVVFSKVNQCLLSKYDKKFTPHLRGLVTG |
| | HAD- | MPKKAAVTYMLEHEKLSGKVDVDEYCKKYDEMAEEMLPKCSLMPGVMKL |
| | superfamily | VRHLKTHRIPMAICTGATKKEFEIKTRHHKELLDLISLWVLSGDDPAIKRGKP |
| | hydrolase | APDPFLVTMDRFKQKPEKAENVLVFEDATNGVCAAIAAGMNVVMVPDLTY |
| | | MKIPEGLENKINSVLKSLEDFKPESVGLPAYDASSNE |
| 31 | OVOC9988 | IPQRRQQQQQQQQQQQRDEREIPPFLEGAPPSVIDEFYNLLKTDENKTDQ |
| | Serine/ | QTEADVEAFINRLGGSYKVRFTQFMEEVKKARADYERIHQQAVARFSPAA |
| | threonine | KDADARMSAIADSPHLTTRQKSQQIQAIMDSLSESVRREIINALSPQE |
| | protein kinase | |
| | DDB_G0280133 | |
| 32 | OVOC4230 | DLLSEAGDFFTKHFTDIKSLFAKDEKQLQQSVDRVKDLLATIQDKMSMLQP |
| | Conserved | LANDMQKTTLGKIGDLISQVNSFRETMSNPKMDFTNKENKWEELLKKIFVT |
| | secreted | EGLNKVIPLLQKLKNSAPTTFATYLFTCIVPVLINTLRE |
| | protein | |
| 62 | OVOC7453 | KNPSKMESKTGENQDRPVLLGGWEDRDPKDEEILELLPSILMKVNEQSKD |
| | (CPI2M) | EYHLMPIKLLKVSSQVVAGVKYKMDVQVARSQCKKSSNEKVDLTKCKKLE |
| | | GHPEKVMTLEVWEKPWENFMRVEILGTKEV |

Natural immunity against O. volvulus can be acquired in a few individuals in affected populations and these individuals are known as putatively immune. Consequently, they exhibit protective immune response against L3 larvae, suggesting that E/S products released by molting larvae and/or surface proteins of L3 larvae are an important source of protective antigens. The identification of proteins that are highly expressed by the mf and that are specifically recognized by sera from protected individuals who never developed a patent infection opens up new possibilities for also developing a safe anti-transmission or therapeutic vaccine. The identification of Ov-unique proteins that are adult and/or mf stage-specific that are recognized by sera of Ov-infected individuals provided additional novel biomarkers needed for better mapping the prevalence of infection and for post-control surveillance.

It is anticipated that O. volvulus proteins, or orthologs thereof, will provide protection against infection with D. immitis. D. immitis orthologs of O. volvulus proteins are provided in Table 5.

TABLE 5
D. immitis orthologs of O. volvulus proteins

| SEQ | Protein | | |
|---|---|---|---|
| ID NO. | Description | Ortholog of | Sequence |
| 33 | nDi.2.2.2.t | OVOC8619 | MRTASQLTFMLFLVLKKKFKNIDKLFSQISVNMSVMFANSRS |
| | 00004 | | SQANSGYLVEFKAGRSNLQAGSTVDKRKVVADKTKGLIFIKQ |
| | Proteasomal | | SSDQLMHFCWKNRETGTVVDDLIIFPGDTEFLRVKECTDGRV |
| | ubiquitin | | YMLKFKSTDEKRLFWMQDGKTDKDDENCKKINETLNNPPAP |
| | receptor | | RAAARGGADRAGASSFGTLAALGSAGADSELGALGNLDQN |
| | ADRM1 | | QLMQLLSLMNHTNSASASEAANLLPQLPLVADTPNPVASEES |
| | homolog | | GTTSTQGATPSNTPANGIIAGSSSNNAVQLSQLKEIIASITPPD |
| | | | GSIRKPSVDFTDVLCCADKINDVLGKYAERLIPHLPNQEPIYN |
| | | | NQEELQQTLRTPQFRQAVDIFGHALQTGQLAPILRQFGIDSN |
| | | | TAIAAGNGDLIAWATQFTTSENEKEIAVKTETLPFHPGMESDV |
| | | | EDEETNEKAVRESDKNRTDDHMDLD |
| 34 | nDi.2.2.2.t | OVOC7083 | MLPTLYINNAVIRPVLSETKKVKVQNISSPFLIFLLLSITKMLSLS |
| | 03357 | | VLLLFISMATMARPPNPDEIKELHEQQLNDSKDDYDMLPDVG |
| | | | HIPESFKESLKKQKMLYLDMLRQQSL |
| 35 | nDi.2.2.2.t | OVOC4111 | MISSRLRITIPESIVIFGIFCFFIFFCFLSFFFFFTLWSHRDTINFQ |
| | 05919 | | TDFMTETIKFIVYAVVILRMMFFDIVCFYSFLMMTIVLINTSNGL |
| | | | SVPAGLRPAKKVGDPREQIVPGKEQQQQREQQQQQQQQLQ |
| | | | EEEQQQQQQHDEVSNLRPTPKVPPNLSIRSRMMAALSASPV |
| | | | EPNKEKNSSKVETDSFSKPPIIFSKGNKKTVPGKIAPSGSSKG |
| | | | NARVIVAPPADLGKNNYGLNTVLQTNLVDSHGRIMKNVNSVPI |
| | | | KVPSSAEMKNARTRHTARQVESDADKVVPIKFGSTSRRR |
| 36 | nDi.2.2.2.t | OVOC1808 | MMRIKWIILLLLLLLPIITAEFSAPVGTNSSLTIFDKDKQVLLRSD |
| | 07753 | | RLKRQCGPCGVAPSPVIVCCGAAGLKEIFRSWWLHIPLLLLP |
| | | | MSTSWLKTMVC |
| 37 | nDi.2.2.2.t | OVOC11598 | MFRLLIAIQILRFCQANYINDVYWKRSIIGYQHIPIILNICYLLQTE |
| | 06812 | | VSNKGVVDALFLHSPTYHRVEMSEETDNIESIADKSNITVANKP |
| | | | NLMIYPADFQVSSNERASASIPITITITSSGDTIIKSFKHKHQSNE |
| | | | IFKRRVAKMAIAPVNAPEVENLAPEVENPSPSTAGYESKTEEQ |
| | | | APSESGQYGKRRK |
| 38 | Fibroblast | OVOC3901 | MYNLAKLLENEHGSIEASFHVYVENFFEGLDGESWSIDQTNA |
| | growth factor | | QLYPIIDEPFNNTVRVGRTAQFQCKVKNQQQPLIKWLKRIDDP |
| | receptor-like | | NAIRQANANATLIHANNMHLLLLEKPETSAELSDGISLNRLIIPN |
| | 1 | | VRYEHSGTYLCVVTNAHGDIAYRSAYLHVIARSDHGMLSNIYF |
| | nDi.2.2.2.t | | YGGILVLIVVFTLITYAVYFLRKNQAAKNSESAQDITNTRYSFSL |
| | 10368 | | RPPPPNLPPPKAPALPSERQQLMSDNQPCDRYAVNSAATTYY |
| | | | PQFATPDKKLQKIITESGGTRPTPIRRTNGGDTKYRLKDEYINS |
| | | | PKWVHTKGDNIEVEMDQNLLKNRSSHCYNPISGAYGRTDNID |
| | | | RQQQKSFLTIGNLQKR |
| 39 | nDi.2.2.2.t | OVOC10819 | MLKLANTEIFFIAFLVYSKEIILNCYEDYKECVATSNKTNHVNMD |
| | 02919 | | NVNPQNLIEFCFDHTQNILPCLVTKLGLTKGISVSIFSLFLSTCE |
| | | | LEAQNNKSSSTTEMQQILRHLLRLYAYFCAYSNIIDLHRNRECF |
| | | | RYLMKRCVLNKPDESCMFYHCGKIHFNLSKSSRKILFTRQHDT |
| | | | TKIVNLGNKMNQLATFNNHQVRSAVVVTLIITFIDMIQ |
| 40 | nDi.2.2.2.t | OVOC5395 | MYSQENQDDKRRNDERIALSVPYNNTNIMDRSYFKPIKLSYPY |
| | 01093 | | SDECLEVNKKSSDDSDQRLSQNSSTPQSNYDQSSERLSIDDS |
| | | | DLIDDSTSAAQLSTSSPISVTSASTSSFYPTLNIGNGMEMNAKY |
| | | | AKIEQSEGIRSDQSSTLRISDKYKKYTAIKRRLSELCGIIEEKDK |
| | | | QLRVVRNGLNEKDLEIGKLCDKIRALEYNCGRLQAVIESVGDE |
| | | | SDQNQIKLHEIINERDGLLVRNASLSRQIEFEKREWSIERERLS |
| | | | MDLDDVTRELELQKMILNGENISEIVQRWQTKVFELEGMIADR |
| | | | DRAIRAQQVRISKLKQSLAEADRISCDDSSESQTKLDSPSFTCI |
| | | | KRLLLQYLTSSDEERIQLLRNVSTMLHLSDDEQHQVLTNLKSRI |
| | | | QIS |
| 41 | nDi.2.2.2.t | OVOC12235 | XKCRDQRAEVEYVNGSAKNCLYWPCTVGYFCEYTESRNGGH |
| | 11596 | | YICCGTNANNIYGKVKVYPGTNKPLHCSIMNTCPFLDTPNCVM |
| | | | SHRYGYKVCCSTMNC |
| 42 | nDi.2.2.2.t | OVOC7908 | MLMKQSDSCVDYFYDQYKGQEYVKDDAFNTQNITDNFRKSS |
| | 05701 | | SDIAQLMNSQIELISQPEKVNEDSAKSSHYNDDLQKSIEDDTVE |
| | | | ATQRKKDEKLLEFLHSLIVSTIPKTIHLEGNSVNLLTLTTTITPIAII |
| | | | TTKNTSGTANAITTRKYKKYKLNAFVNISSDTLTELPKFLPENF |
| | | | NSTNFANVEKTEKFSNSKQVATDSIFSLKESAYLETPVIRDFSS |
| | | | ANDSAKTDPLFTRNYVDKQIDMNTTKFNKNLKKSRLTTISTSNL |
| | | | TTVLSQLQTTTSISTTTSVTTTISTSITIPELTLVSQSHRHLHHYH |
| | | | HHHHHQYENYDHESPIIVTALFDIGRGKWPRYTRTYEQYMNY |
| | | | LKHLLKLENCLVIYTDSRGAEFVRQTRNVHNTQIFEISMHDLPL |
| | | | YRYREEMKGIIQREQKDWQFSPKTRYHPEANSADYNIIVNSKP |
| | | | YFLYNATQNVRFRTSDRMFVWIDAGYGHGRKGIIPDHCHWRP |
| | | | RLQRDRMTIIQLTPKHDKVSRYSITDLYRVDWVVLSGGFIAGD |
| | | | SHTINRFYRFYQKLFMELLDSGRIDDDQTILTLMLKHYTTLFNPI |
| | | | SSNGDWYALFRLFPCHDRQ |
| 43 | nDi.2.2.2.t | OVOC7430 | MKQATTWGSICEMCPCAAKPICPPPVICPPRICPPPVICPPQIC |
| | 04336 | | PPCPPRICPPPVICPPQICPPCPPQICPPCPKPQPPPPPPPPPV |
| | | | LPSLPPTSFKPMITCCRTCICYIRRKRDSLNDYDRIHDINPVCN |
| | | | NDQLMMIMKKKIRTNVTESTIAIKKAADSMLQAEFNVFCAINDL |
| | | | THVAHAEHFCQYKKDNSVFDSFLFRSTLKGLIEECREGVRWW |
| | | | PGSLGDLDFSHISLYRAHKYIGNEEMNRSTKTKISFTRINKKWR |
| | | | LGHTGKKYNKVRFSRNIAKKFIGVCNIIRLKKSVSRSVRPFENQ |
| | | | KSTSFNVFQLLVPKEKVEIVVDDTQAEEMNSETAQEVQLFNVR |
| | | | KSNADSKTDGEKDTADLDVILLTNEECSSSRQENLNKDEPEIVI |
| | | | LDDSAPSKSDLNTSDEIICLQDLKMVNEVPTFSVTPKQKTVKEL |
| | | | PRETRTYGTRRGRQSRAYCEDLRKFPSIRNPVSSSSSSIHAKN |
| | | | MPEFVDLLTQGTLLICKKWLRRWDIVQSGVIGGNPLRICSYNV |
| | | | LCQQTAYKTPELYIHLTKPGRAYELTWENRWRLLTREFSMIGA |
| | | | DIFCLQEVQYDHYDQFFRPYFEAAGFFGKYKKRTNNLLDGCAI |
| | | | FYKSHLQLLHYRYIEYFLNIDSVLNRDNVGQLIRLKDMRSGREF |
| | | | CVVNTHLLFNKRRGDVKLAQLAILLANIDQECGPESGQECPYIL |
| | | | CGDFNFHPYSPIYNFIMNGEICFTNLRRGDISGQGNAGGPFVS |
| | | | VNLLPEDVKIARNCRFNYLKNRTMLLPSLNCWSHPLCFNSVY |
| | | | QNMNGETRPMISTYHSIEAVNPDFIFYSVKSKRVQQSMLPHSV |
| | | | PAMNVSEREIRLIRRLSLPDMNELAGTLGPWPNSTTPSDHIPLI |
| | | | ADFVLQ |
| 44 | nDi.2.2.2.t | OVOC8936 | MYCKLIISFYMLLSIANMTHLVGYRPQIYLQGIPQNIQSHDIQRL |
| | 10647 | | DMQQQSLKLPDTELYSIPSHDNQLQGLQLYDMQFQGKQSKG |
| | | | SEKLCSGCKISINCSGKKCVPMRTRKPIVTTPSPLSTQRPVLTR |
| | | | PRLLADCPCPCHVSRQCRICQPCQESFI |
| 45 | nDi.2.2.2.t | OVOC5806 | MFVGMRLYLAIDVLLLLVLRIKSNRIILHRFSLFIQQHCCNNISQI |
| | 03537 | | HRLNDCKYSKVRMKIDKKILIIVSKTEWCNEAIKVVFGKSAEAR |
| | | | RNRSDAISWVTPYNFTGLMNLHSKWRYDAYFDNVTRTAHGLA |
| | | | RIDLLCPKRRSHSGRRILKRSIQENKQEKSRRSFTVNIYGSSKG |
| | | | IFTITVGVVIYAIFGVCFLITNMAYLSGIYTVHNTSVIPEDKKRKE |
| | | | TSKRKEIL |
| 46 | nDi.2.2.2.t | OVOC4665 | MISVFLLLTVIVSYVETIGENPMDINALAGIIGGISNMMQNNVETI |
| | 01073 | | DVPSSQIMGQWYQVYKAAISFDAYKTDMFCPVAYFKPNSVMG |
| | | | EDGFSIEEAYRVITKNGPVETFKRDLNKVGTGQYWMYTEEYF |
| | | | YPRQFNIIGVGPNYTNATDGREKENLYEYMIVTDANRLSLSVY |
| | | | ARHPMIFYQKYNEEVVKFLEHAGFGGRVFWNSPRPIYQGTDC |
| | | | EWPSEKEVFARRVLKNQEAARNTGLETATKSGLFGSSLTTDA |
| | | | YNPIKEMLQNPQLALQKLVQGH |
| 47 | nDi.2.2.2.t | OVOC8227 | MTVIKSMLNITHVIFDLDGLLINTEIVFSQVNQCLLSKYGKKFTS |
| | 00378 | | HLRGLVTGMPKKAAVAHILEHERLSEKIDVDEYCKKYDEMAEE |
| | | | MLPKCSLMPGVMKLVRHLKAHSIPMAICTGATKKEFELKTRCH |
| | | | KELLDLISLRVLSGDDPAVKRGKPAPDPFLVTMERFKQKPEKA |
| | | | ENVLVFEDATNGVYAAIAAEESKIVK |
| 48 | nDi.2.2.2.t | OVOC9988 | MILEQLEVPPFLVGAPQSVIKQFYDLLKADETKTDAQTEADVE |
| | 01674 | | AFINRLGGTYKTRFDQFKQEIKQGKAAYERLHQQAVAKFSKEA |
| | | | READAKMSAIADSPSLTTQQKTQQIQAIMD |
| 49 | nDi.2.2.2.t | OVOC4230 | MLKYGILLILITVGAYCDLLSEAGDFFSKHFTDFKSLFASDEKQL |
| | 06953 | | QQNMDRVKDLLATIQDKMTILKQLADNSQKSTLEKITDIISQVN |
| | | | DFRENVFNSNVDFNQKKTKWEEVVTKIFVTDGLNKVIPLLQKA |
| | | | KNSAPATFITYLLTCIVPLLINALRE |

### Example 5. Immunoreactivity of O. volvulus Proteins

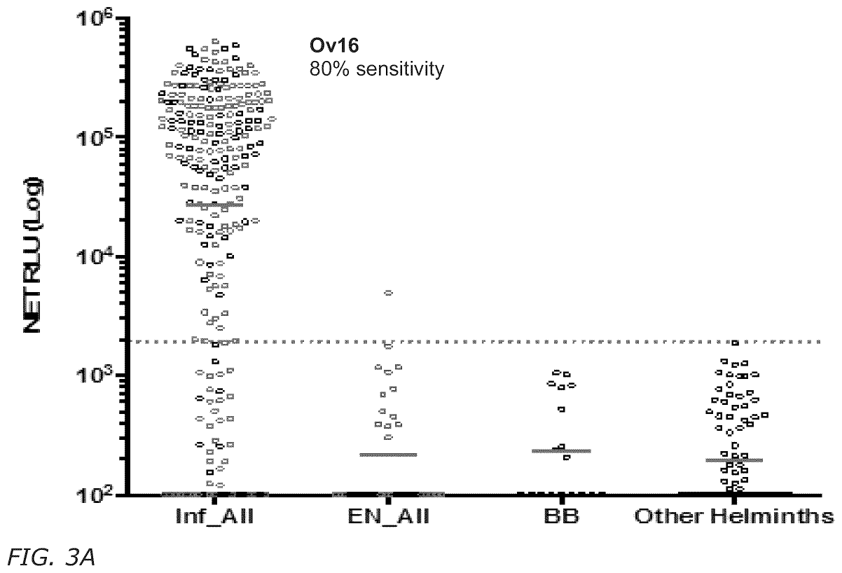

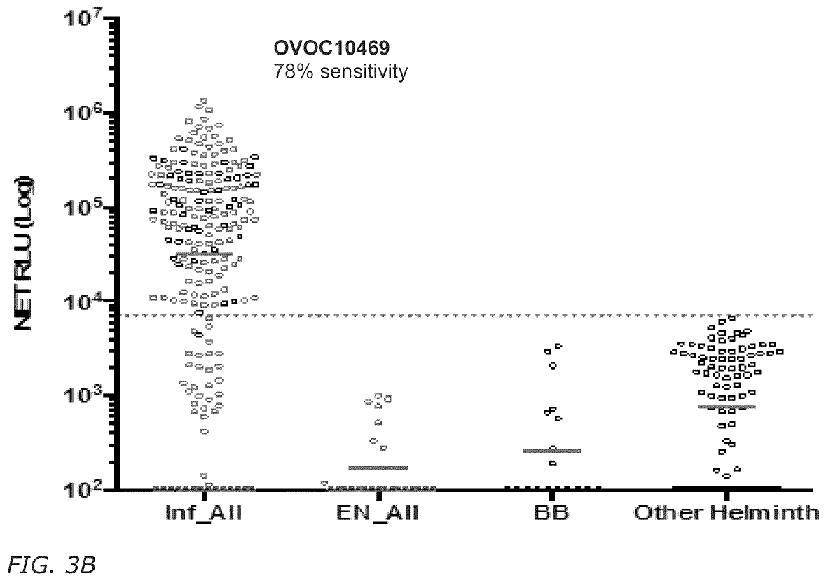

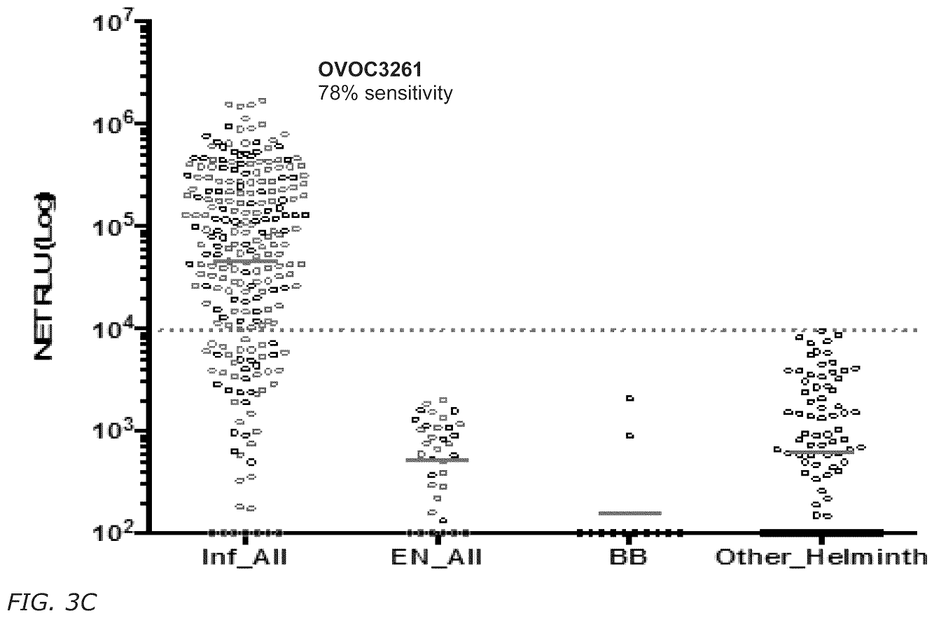

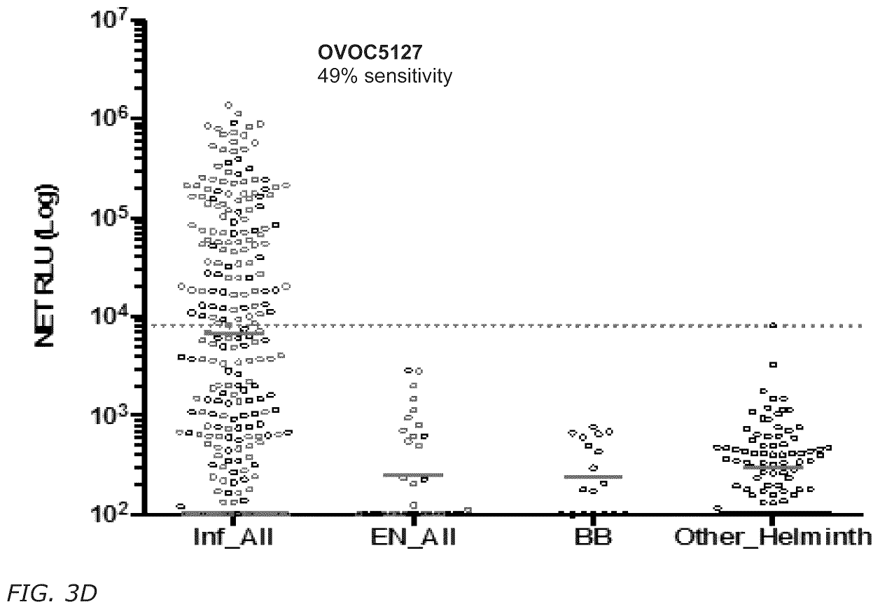

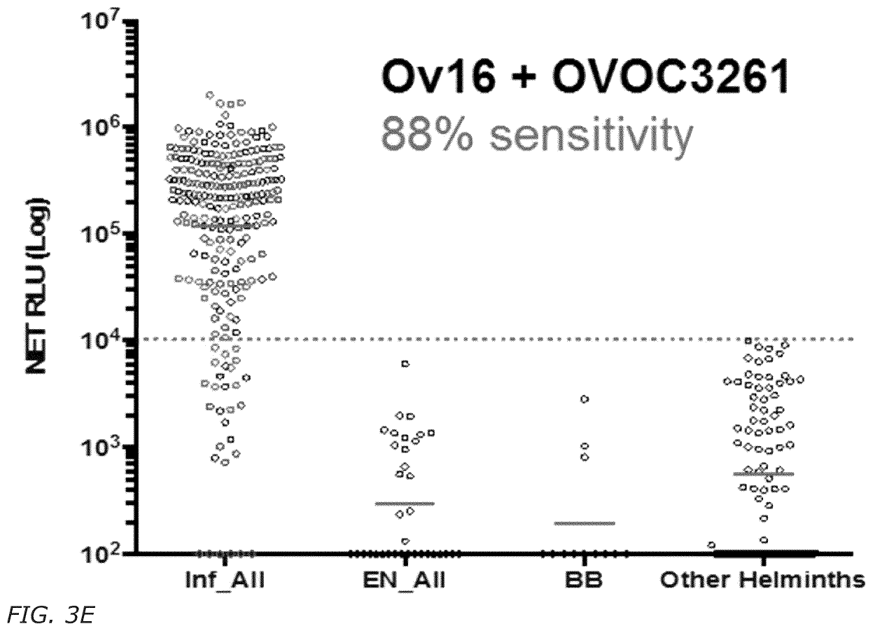

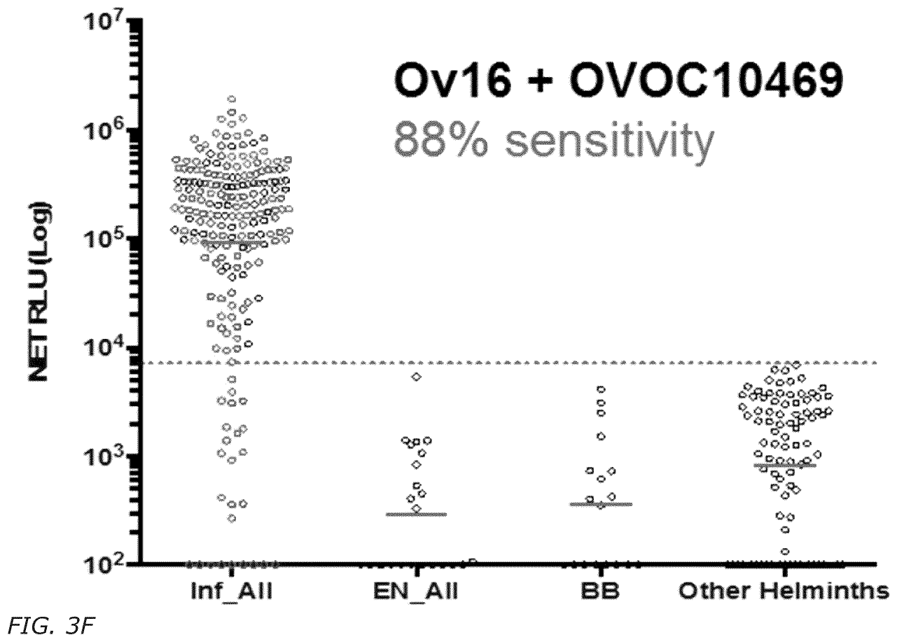

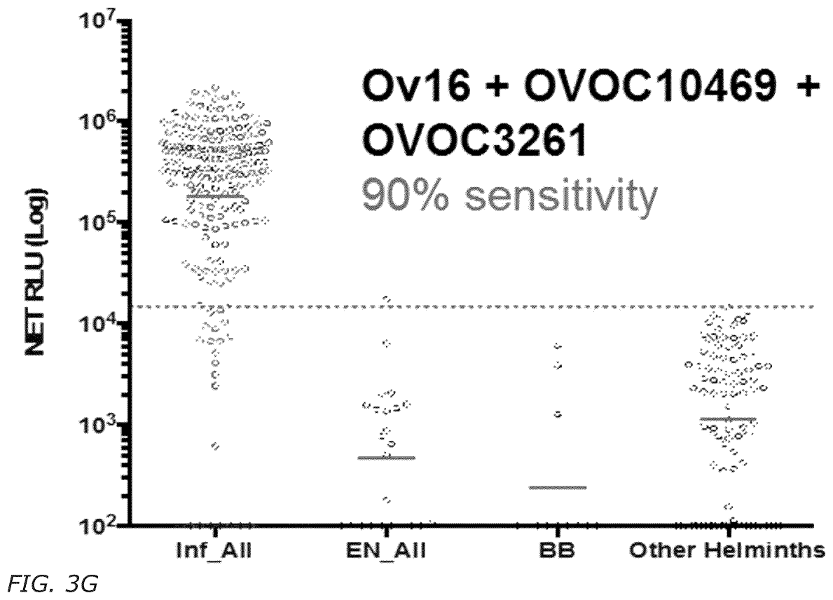

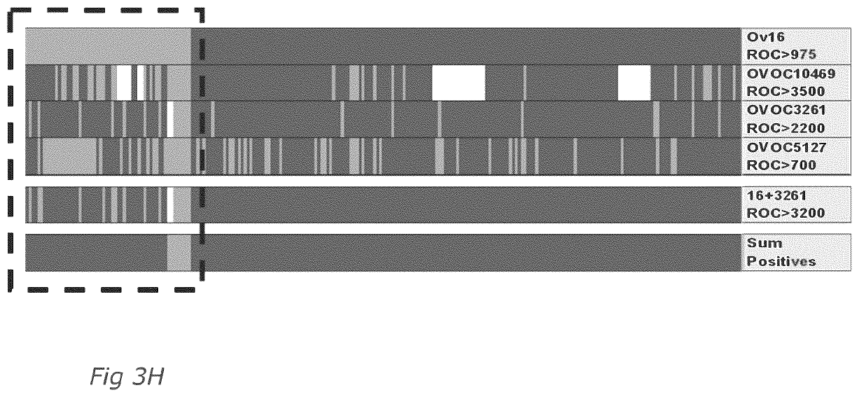

Among the biomarker sequences listed in Table 3, OVOC3261, OVOC5127 and OV10469 were tested for their individual immunoreactivity using a variety of Onchocerca microfilaria positive infected sera (truly infected) and a variety of control sera (non-infected (EN_all, BB), infected with unrelated human filarial pathogens or S. stercoralis) in immunoassays. As can be seen in FIG. 3, when using a cutoff that gave 100% specificity, OVOC10469 had 78% sensitivity (FIG. 3B), OVOC3261 had 78% sensitivity (FIG. 3C), and OVOC5127 had a sensitivity of 49% (FIG. 3D). Combinations of these newly identified proteins were tested in combination with the known Ov16 (KISAENANCKKCTPMLVDSAFKEHGIVPDVVSTAPTKLVNVSYNNLTVNLGNELTPTQ VKNQPTKVSWDAEPGALYTLVMTDPDAPSRKNPVFREWHHWLIINISGQNVSSGTVLS DYIGSGPRKGTGLHRYVFLVYKQPGSITDTQHGGNRRNFKVMDFANKHHLGNPVAGNF FQAKHED; SEQ ID NO:63) (Table 6), it can be seen that the sensitivity increases for all of these combination compared to Ov16 alone. Each individual positive sera was tested against each of the antigens and in combination with all four. As can be seen in Table 6, the combination gets to 97 percent sensitivity (8/245 mf positives being false negatives).

TABLE 6
Reactivity of Ov-infected samples with O. volvulus biomarkers

| | | | | | | Combination |
|---|---|---|---|---|---|---|
| | | | | | Ov16 + | of 4 |
| | Ov16 | OVOC10469 | OVOC3261 | OVOC5127 | OVOC3261 | biomarkers |
| Positive | 187 (77%)* | 167 (81%) | 219 (91%) | 172 (70%) | 225 (94%) | 235 (97%) |
| Negative | 53 (23%) | 40 (19%) | 22 (9%) | 71 (29%) | 16 (6%) | 8 (3%) |
| Not tested | 2 | 38 | 4 | 2 | 4 | 2 |

*number of samples (percent of samples tested)

Further analysis demonstrated that OVOC3261, OVOC10469, OVOC8491, OVOC11950, OVOC10602 are microfilaria-specific. Moreover, most of these antigens are relatively invariant based on non-synonymous SNPs and that antibodies of the IgG and IgG₄ isotypes of two of these (OVOC3261 and OVOC10469) only appear after microfilariae appear in the skin of experimentally infected chimpanzees.

### Example 6. Immunization of Mice with O. volvulus Proteins

Yeast codon optimized DNAs encoding for O. volvulus proteins Ov-CPI-2M (OVOC7453), Ov-103 (OVOC4230), and Ov-RAL-2 (OVOC9988), minus the signal peptides at the N-terminus, were synthesized and subsequently subcloned in-frame into the yeast expression vector pPinkα-HC (LifeTechnologies) with XhoI/KpnI sites and E. coli expression vector pET41a (EMDMillipore) with the fusion GST deleted (NdeI/XhoI). The correct open reading frame (ORF) was confirmed by double-stranded sequencing using the vector flanking primers (5′AOX1/CYC1 for pPinkα-HC and T7 promoter/T7 terminator for pET41a). For expression in yeast, the recombinant plasmids were linearized with AfIII digestion and then transformed by electroporation into PichiaPink strain #4 with protease A and B knockout (pep4/prb1⁻) to prevent P. pastoris-derived protease degradation. Yeast transformants were selected on P. pastoris adenine dropout (PAD) selection plates. The expression of recombinant filarial antigens with hexahistidine (6His)-tag at the C-terminus was induced with 0.5% methanol and the soluble recombinant proteins secreted into the culture were purified with immobilized metal ion affinity chromatography (IMAC). For expression in E. coli, the recombinant constructs cloned into pET41a were transformed into BL21(DE3) (EMDMillipore) and recombinant proteins were induced with 1 mM isopropyl-β-thiogalactoside (IPTG) and purified with IMAC.

In order to test the synergistic protection of two or three O. volvulus protective antigen combinations, protective O. volvulus antigens Ov-103, Ov-RAL-2 and Ov-CPI-2M, were fused together as a triple antigen (Ov-103-RAL-2-CPI2-M) or as two double antigens (Ov-103-RAL-2 and Ov-RAL-2-CPI2-M) by using a flexible linker (KGPDVPETNQQCPSNTGMTD; SEQ ID NO:50) obtained from Na-ASP-1 structure between two pathogenesis-related (RP) domains. The yeast codon optimized fusion DNAs were subcloned into either yeast expression vector pPICZUαA (Life Technologies) or E. coli expression vector pET41a with GST knockout. The recombinant fusion proteins were expressed and purified using the same methods described above except for the use of yeast strain P. pastoris X-33.

TABLE 7

| SEQ ID | Protein | |
|---|---|---|
| NO. | Description | Sequence |
| 64 | Ov-103- | DLLSEAGDFFTKHFTDIKSLFAKDEKQLQQSVDRVKDLLAT |
| | RAL2- | IQDKMSMLQPLANDMQKTTLGKIGDLISQVNSFRETMSNP |
| | CPI2M | KMDFTNKENKWEELLKKIFVTEGLNKVIPLLQKLKNSAKG |
| | fusion | PDVPETNQQCPSNTGMTDPQRRQQQQQQQQQQQRDEREIPPF |
| | protein | LEGAPPSVIDEFYNLLKTDENKTDQQTEADVEAFINRLGGSYK |
| | | VRFTQFMEEVKKARADYERIHQQAVARFSPAAKDADARMSAI |
| | | ADSPHLTTRQKSQQIQAIMDSLSESVRREIINALSPQEKGPDVPE |
| | | TNQQCPSNTGMTDKNPSKMESKTGENQDRPVLLGGWEDRDPK |
| | | DEEILELLPSILMKVNEQSKDEYHLMPIKLLKVSSQVVAGVKYKMD |
| | | VQVARSQCKKSSNEKVDLTKCKKLEGHPEKVMTLEVWEKPWENF |
| | | MRVEILGTKEV |
| 65 | 0v103- | DLLSEAGDFFTKHFTDIKSLFAKDEKQLQQSVDRVKDLLAT |
| | RAL2 fusion | IQDKMSMLQPLANDMQKTTLGKIGDLISQVNSFRETMSNP |
| | protein | KMDFTNKENKWEELLKKIFVTEGLNKVIPLLQKLKNSAKG |
| | | PDVPETNQQCPSNTGMTDPQRRQQQQQQQQQQQRDEREIPPF |
| | | LEGAPPSVIDEFYNLLKTDENKTDQQTEADVEAFINRLGGSYK |
| | | VRFTQFMEEVKKARADYERIHQQAVARFSPAAKDADARMSAI |
| | | ADSPHLTTRQKSQQIQAIMDSLSESVRREIINALSPQE |
| 66 | OvRAL2- | PQRRQQQQQQQQQQQRDEREIPPFLEGAPPSVIDEFYNLLKTD |
| | CPI2M | ENKTDQQTEADVEAFINRLGGSYKVRFTQFMEEVKKARADYE |
| | fusion | RIHQQAVARFSPAAKDADARMSAIADSPHLTTRQKSQQIQAIM |
| | protein | DSLSESVRREIINALSPQEKGPDVPETNQQCPSNTGMTDKNPSK |
| | | MESKTGENQDRPVLLGGWEDRDPKDEEILELLPSILMKVNEQSKD |
| | | EYHLMPIKLLKVSSQVVAGVKYKMDVQVARSQCKKSSNEKVDLTK |
| | | CKKLEGHPEKVMTLEVWEKPWENFMRVEILGTKEV |

Ov103 sequence - Bold; Linkers - Highlighted; OvRAL2 sequence - underlined; OVCPI-2M sequence - Italics.

The purity and the molecular weight of purified recombinant proteins were analyzed by SDS-PAGE using pre-cast 4-20% Tris-glycine gels (Life Technologies) and stained with Coomassie brilliant blue R-250 (Fisher Scientific).

Male BALB/cByJ mice were purchased from The Jackson Laboratory at 6-8 weeks of age. All mice were housed in micro-isolator boxes in a room that was pathogen-free and under temperature, humidity and light cycle controlled conditions. Mice were fed autoclavable rodent chow and given water ad libitum. All protocols using mice were approved by the Institutional Animal Care and Use Committee.

Mice were immunized with 25 μg of the produced vaccine antigens (0 v-CPI-2M, Ov-103, or Ov-RAL-2, or the two- or three-antigen fusion proteins) in 0.1 ml of Tris-buffered saline (TBS) formulated with 0.1 ml of 1:5 Rehydragel LV (alum) in PBS (General Chemical). Mice were immunized s.c. in the nape of the neck, followed by two booster injections 14 and 28 days later.

The mice were challenged 14 days after the final booster as previously described (Hess et al., Int. J. Parasitology 44:637-646, 2014) with 25 L3 larvae delivered within a diffusion chamber. The diffusion chambers were implanted in a s.c. pocket on a rear flank of each mouse. Recovery of the chambers was performed 21 days later and larval survival was determined based on mobility and morphology of the remaining larvae. Protective immunity was calculated in two ways: (i) percentage of reduction in larvae was calculated as follows: % reduction=((average worm survival in control mice−average worm survival in immunized mice)÷average worm survival in control mice)×100; and (ii) host protection was calculated as follows: (number of immunized mice with parasite recovery levels below the lower S.D. of parasite recovery in control mice÷total number of immunized mice)×100). Host cells within the diffusion chamber were collected and analyzed by centrifugation onto slides using a Cytospin 3 (Shandon Inc.) and then stained for differential cell counts using Hemastain 3 (Fisher Scientific).

Serum was collected at the time of recovery for antigen-specific IgG analysis. Maxisorp 96-well plates (Nunc Nalgene) were coated with 2 μg/ml of the immunizing recombinant antigen in 50 mM Tris-CI coating buffer, pH 8.8, overnight at 4° C. Plates were washed with deionized water between each step. Plates were blocked with borate buffer solution (BBS) (0.17 M boric acid, 0.12 M NaCl, 0.5% TWEEN 20, 0.025% BSA, 1 mM EDTA, pH 8.2) at room temperature for 30 min. Individual sera were diluted to an appropriate starting concentration with BBS and serially diluted; plates were sealed and incubated at 4° C. overnight. Biotinylated IgG (eBioscience) was diluted 1:250 in BBS and incubated for 1 hr at room temperature, followed by ExtrAvidin Px (Sigma) which was diluted 1:1000 in BBS and incubated for 30 min at room temperature. One component ABTS peroxidase substrate (KPL) was added and O.D.s were read after 30 min at 405 nm in a Bio-RadiMark Microplate reader (Bio-Rad). ELISA data are presented as endpoint titers which were calculated as the serum dilution from experimental animals that had an O.D. reading three times higher than the O.D. recorded for control serum.

Onchocerca volvulus proteins were expressed as soluble recombinant proteins in high yield in P. pastoris and E. coli BL21(DE3) after being induced with 0.5% methanol for P. pastoris and 1 mM IPTG for E. coli, and purified with IMAC. Purified recombinant Ov-103, Ov-RAL-2 and Ov-CPI-2M expressed in P. pastoris or in E. coli migrated at the same molecular mass as calculated by the coding sequence (14.5 kDa, 17.9 kDa and 16.0 kDa, respectively) on SDS-PAGE and Coomassie staining. The fusion recombinant proteins of two or three antigen combination (Ov-103-RAL2, Ov-RAL2-CPI2M and Ov-103-RAL2-CPI2M) were also expressed in P. pastoris and E. coli expression systems as soluble proteins and the purified recombinant fusions were shown at the correct molecular weight as estimated by sequences on SDS-PAGE (50.6 kDa, 32.5 kDa and 35.2 kDa, respectively).

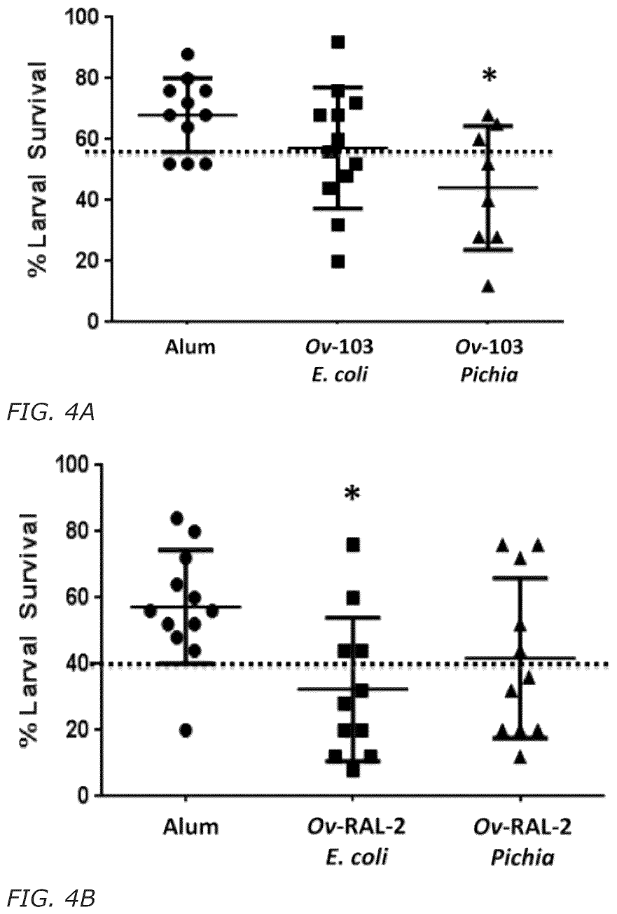

In BALB/cByJ mice immunized with Ov-103 with alum prepared in both P. pastoris and E. coli expression systems, E. coli-expressed protein induced an 8% reduction in larval survival and a 50% level of host protection, whereas mice immunized with the P. pastoris-expressed protein had a statistically significant 30% reduction in parasite survival and a 63% level of host protection (FIG. 4A). Differential cell counts were performed at the conclusion of the experiments on the diffusion chamber contents. Comparable numbers of total cells (1.4×10⁶±1.3×10⁶), and percentages of lymphocytes (5±7%), neutrophils (52±20%), macrophages (37±15%) and eosinophils (12±14%) were seen in the control and immunized mice. Parasite-specific antibody titers show equivalent endpoint titers for mice immunized with P. pastoris and E. coli expressed Ov-103 when measured against both the P. pastoris and E. coli expressed proteins (Table 8). Correlation analyses were performed between parasite survival and antibody endpoints titers and there were no significant relationships between the amount of antibody produced and the survival of the larvae.

Mice immunized with E. coli-expressed Ov-RAL-2 induced a statistically significant 39% reduction in larval survival and a 64% level of host protection, whereas mice immunized with the P. pastoris-expressed protein induced a 24% reduction in parasite survival and a 55% level of host protection (FIG. 4B). As with Ov-103, differential cell counts showed comparable numbers of total cells, lymphocytes, neutrophils, macrophages and eosinophils in the control and immunized mice. Parasite-specific antibody titers show equivalent endpoint titers for both the P. pastoris and E. coli expressed proteins (Table 8). Again, correlations between parasite survival and antibody endpoints titers did not reveal any significant relationship between the amount of antibody produced and parasite survival.

Immunization of mice with Ov-CPI-2M expressed in both E. coli and P. pastoris induced statistically significant reductions of 30% in larval survival and 17% levels of host protection (FIG. 4C). As with the other two antigens, differential cell counts showed comparable numbers of total and specific cells in the control and immunized mice, and parasite-specific antibody titers had equivalent endpoints (Table 8). There were no significant correlations between antibody endpoint titers and parasite survival.

TABLE 8
Geometric mean of IgG endpoint titers following
immunization with individual, fusion, or concurrent antigen formulations.

| | Endpoint titer to antigen | | | | | |
|---|---|---|---|---|---|---|
| | | | Ov- | Ov- | Ov-RAL- | Ov-RAL- |
| | Ov- | Ov- | CPI- | RAL- | 2/CPI- | 2/103/ |
| Immunizing Antigen | 103 | RAL-2 | 2M | 2/103 | 2M | CPI-2M |
| Ov-103 E. coli | 33,064 | | | | | |
| Ov-103 P. pastoris | 35,882 | | | | | |
| Ov-RAL-2 E. coli | | 571,055 | | | | |
| Ov--RAL-2 P. pastoris | | 519,490 | | | | |
| Ov-CPI-2M E. coli | | | 431,803 | | | |
| Ov-CPI-2M P. pastoris | | | 462,057 | | | |
| Ov-103/RAL-2 fusion | 317,320 | 439,250 | | 1,509,278 | | |
| Ov-RAL-2/CPI-2M | | 187,884 | 266,079 | | 691,063 | |
| fusion | | | | | | |
| Ov-RAL-2/103/ | 90,464 | 146,607 | 165,510 | | | 1,112,542 |
| CPI-2M fusion | | | | | | |
| OV-RAL-2, 103, | 16,019 | 271,416 | 392,676 | | | |
| CPI-2M concurrent | | | | | | |

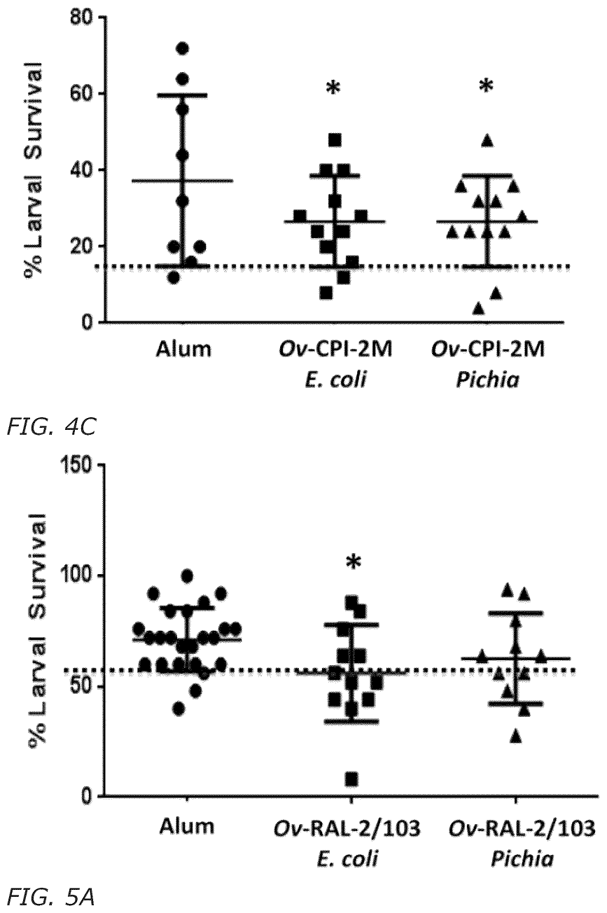

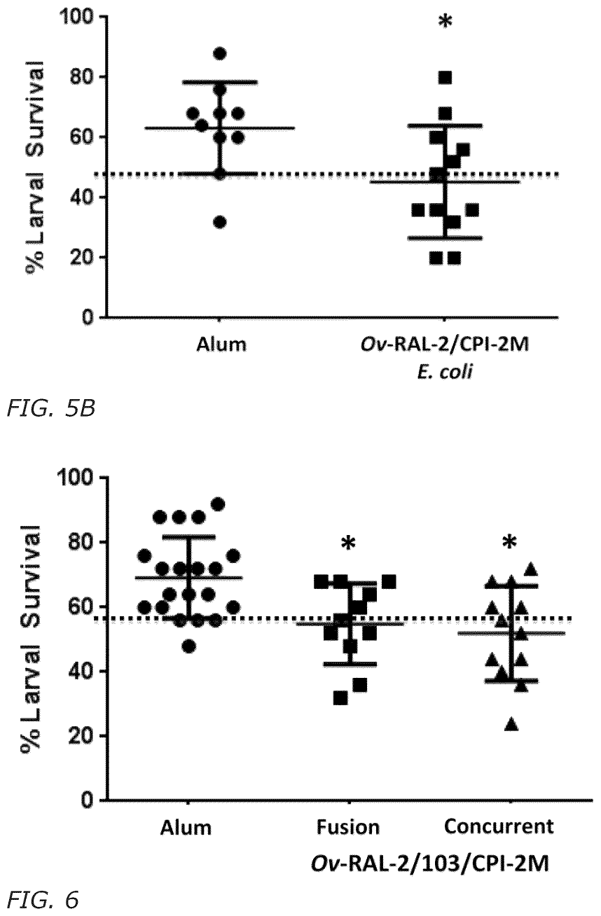

In mice immunized with Ov-RAL-2/103 fusion protein expressed in P. pastoris and E. coli, E. coli-expressed protein significantly reduced larval survival by 21% and provided a 58% level of host protection, whereas immunization with P. pastoris-expressed protein only reduced larval survival by 11% and provided a 45% level of host protection (FIG. 5A). Immunization with Ov-RAL-2/CPI-2M E. coli fusion protein induced protective immunity with parasite reduction at 34% and a 50% level of host protection (FIG. 5B). Analysis of the cells within the diffusion chamber contents showed similar numbers of total cells, lymphocytes, neutrophils, macrophages and eosinophils. Parasite-specific antibody titer endpoints were measured against the individual antigens and the fusion protein. Antibody endpoint titers for the two fusion proteins were significantly higher than the antibody responses in these mice to the individual antigens of which the fusion was composed. The antibody response to Ov-RAL-2 and Ov-CPI-2M by mice immunized with these antigens as part of a fusion were equivalent to the responses seen in mice immunized with antigen individually. However, the parasite-specific antibody titer endpoint to Ov-103 was approximately eight-fold higher in mice immunized with the antigen as part of a fusion compared with immunization with the individual antigen (Table 8). Once again, there were no significant correlations between antibody endpoints and parasite survival.

A fusion protein consisting of Ov-103, Ov-RAL-2 and Ov-CPI-2M was created to determine whether enhanced protective immunity would be achieved with this triple fused antigen. The Ov-RAL-2/103/CPI-2M E. coli fusion was tested in comparison with concurrent immunization consisting of the three antigens injected simultaneously but at different locations on the mice. Immunization with the three-antigen fusion protein and the concurrent immunization resulted in significant levels of protective immunity, with the fusion inducing a 20% reduction in larval survival and a 45% level of host protection and the concurrent immunization resulting in a 25% reduction in parasite survival and a 64% level of host protection (FIG. 7). Analysis of the cells within the diffusion chamber contents showed similar numbers of total cells, lymphocytes neutrophils, macrophages and eosinophils. Antibody titer endpoints were measured against the individual antigens and the fusion protein. Mice immunized with the three antigens concurrently had antibody endpoint titers to the three antigens that were comparable with those seen in mice immunized with the three individual antigens (Table 8). Mice immunized with the three-antigen fusion protein had endpoint titers to the single antigens that were comparable with the titers seen in mice immunized with individual antigens. Antibody endpoint titers for the three-antigen fusion protein were significantly higher than the antibody responses in these mice to the individual antigens of which the fusion was composed (Table 8). There were no significant correlations between antibody endpoints and parasite survival.

### Example 7. Orthologs of O. volvulus Proteins Induce Protective Immunity to Other Filarial Parasites

Brugia malayi is a filarial parasite, one of the three causative agents of lymphatic filariasis in humans. Lymphatic filariasis, also known as elephantiasis, is a condition characterized by swelling of the lower limbs. The B. malayi Bm-103 and Bm-RAL-2 proteins are orthologous to O. volvulus Ov-103 and Ov-RAL-2 which are candidates for development of an O. volvulus immunogenic composition (Table 9). The B. malayi gerbil model was used to confirm the efficacy of these O. volvulus orthologs, alone or in combination, against adult worms. Efficacy of recombinant Bm-103 and Bm-RAL-2 administered individually, concurrently, or as a fusion protein were tested in gerbils using alum as adjuvant. Immunization with Bm-103 resulted in worm reductions of 39%, 34%, and 22% on 42, 120 and 150 days post infection (dpi), respectively, and immunization with Bm-RAL-2 resulted in worm reductions of 42%, 22%, and 46% on 42, 120, and 150 dpi, respectively. Immunization with a fusion protein comprised of Bm-103 and Bm-RAL-2 resulted in improved efficacy with significant reduction of worm burden of 51% and 49% at 90 dpi, as did the concurrent immunization with Bm-103 and Bm-RAL-2, with worm reduction of 61% and 56% at 90 dpi. Immunization with Bm-103 and Bm-RAL-2 as a fusion protein or concurrently not only induced a significant worm reduction of 61% and 42%, respectively, at 150 dpi, but also significantly reduced the fecundity of female worms as determined by embryograms. Elevated levels of antigen-specific IgG were observed in all immunized gerbils. Serum from gerbils immunized with Bm-103 and Bm-RAL-2 individually, concurrently, or as a fusion protein killed third stage larvae in vitro when combined with peritoneal exudate cells.

Thus, immunization with Bm-103 and Bm-RAL-2 individually conferred protection against B. malayi infection in gerbils.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1.-27. (canceled)

28. A method for determining whether a subject has been infected with Dirofilaria immitis, comprising:
(i) providing a composition comprising one or more isolated Dirofilaria immitis proteins selected from the group consisting of a protein having at least 95% sequence identity to the full length mature amino acid sequence of SEQ ID NO: 41, a protein having at least 95% sequence identity to SEQ ID NO: 49, and a protein having at least 95% sequence identity to the full length mature amino acid sequence SEQ ID NO: 48;
(ii) exposing the composition to a specimen obtained from the subject;
(iii) identifying in the specimen from the subject presence of antibodies to the one or more isolated Dirofilaria immitis proteins in the composition, wherein presence of the antibodies indicates that the subject has been infected with Dirofilaria immitis.

29. The method of claim 28, wherein the specimen comprises urine, blood, serum, plasma, or a skin biopsy.

30. The method of claim 28, wherein the subject is a canine subject.

31. The method of claim 28, wherein the antibodies to the Dirofilaria immitis proteins are detected by ELISA, dipstick test, lateral flow, microfluidic device, luciferase immunoprecipitation system, luminex, multiplex-format, or microarray.

32. A composition comprising one or more isolated Dirofilaria immitis proteins selected from the group consisting of a protein having at least 95% sequence identity to the full length mature amino acid sequence of SEQ ID NO: 41, a protein having at least 95% sequence identity to SEQ ID NO: 49, and a protein having at least 95% sequence identity to the full length mature amino acid sequence SEQ ID NO: 48; and at least one epitope tag linked to the one or more isolated Dirofilaria immitis proteins.

33. The composition of claim 32, wherein the epitope tag comprises a histidine tag or a hemagglutinin tag.


SEQUENCE LISTING (66 sequences)

SEQ ID NO: 1 - protein, 98 aa, unidentified
NIAFAPNPKDSNNELFADAESALGSEYAQFVEQSKQHKPVYFSDNQNTLETIKLESIPNPETETAYPMFICGFLGCMKKMNSVEEYLEHFKMHEKQGY

SEQ ID NO: 2 - protein, 103 aa, unidentified
YPTEKETVEPIDTMVKDDIDLVKAEVAEAEEADVEKEVAELTEEEAAEIAEVLDEMEEEFFAFLLFDFILDLFRETLEKNSESQEASIDEVMPEIQGVSAEEA

SEQ ID NO: 3 - protein, 119 aa, unidentified
FRTQSIGIRGRLMCGSKPASNERIKLWEEDSDDLLDQGYTDENGEFLLKGDTVELTPIDPVFKVYHDCDDGIKPGKRKVKFKIPKSYITEGKTPKKIFDLGTLNLETIFNDEERELIVT

SEQ ID NO: 4 - protein, 149 aa, unidentified
SYCEDWDPEDFPSFVLKLSQNATEEFCELYEMEMEVPINKFYDMLRKWAEKYSVQAETNRFIAEEMNYDKMQSKVLMERLQASNGTTEVKGVLEKALKLQESMHLSPDYIQNVIDTMMENLPIDKQNEATLLWNSLYPDDIYNECGPRF

SEQ ID NO: 5 - protein, 426 aa, unidentified
APPNRDTADDLQNADMQRQWEQEQRQREEVQKEEIAKYVKYMRYLETVLNILQATPQWKEAMQSMTQEEMRGGKIAEMVDKLEPHIIEQLAKAKILELQRLEQEIKDQLNADGGATHNIKVSEILTVCLKEGFKKPSNLGIPEHLDFNNWETFSQEDLRKLIVKIVTDMDELEEQRKQDFKQYEMKKKAEEDHKMQAKMIQTEREEYIRQMEEQRRRHNKHEPLKHPGSRNQLRKVWEDTDKLDKDAYDPTTLFGLHDRNNDGYWSYDELNTIFLPEIEKLNNFSDVERLEELYRMRDHVMKQMDTDGDHRISRAEFLADREAQEEKPDQGWEDIGDKDQYTKEELEIFEKEYAKQQGWGEYAYSTPAPTPDPSRMIQPDQAPMQRLDAPSDQVGDMFAQQSHQIPVKHVEPIQSVQQQQMDEVNS

SEQ ID NO: 6 - protein, 231 aa, unidentified
FPTEANPAVGTDNAHEDNLTTEEKMQLKKFAKTNAANFSLTDPEFIDGLKNEAAGLFSKLTGLRDIINAKLDTMQPESRLFIEKLLRRFLAAFSHDGLMNILESLKGFGKEVIDMFDGLSRPIQNDILNAFPLVGSYITSDIARLMLRKLAELDLLSRKSTLTPTVDQFNDDSGKHFPRPQVIEPEEPENSDPEDAQSTDYGKKKVVTTTTFPIITGEEDEILVKKIVENK

SEQ ID NO: 7 - protein, 83 aa, unidentified
IPLPEELDYDGEIPNCRDGEKPLLAADIGVYTCDKNCPKGFRCEYRTMDSTSKKGICCPNLKELAKIYSEDEEVDKSIKKSNI

SEQ ID NO: 8 - protein, 209 aa, unidentified
MISCFALPFPHVCYMAYCTQVIASIMKGWNQNFRFSTVIYLFRNIFSSSVISCVNMILSSTFYALLFVSAVVIVEAMPASESTYSVIIIRINDTTCKIEDGVVSVNGQVIGNLTEEQKEELEAYNVQTQGWFQQLHQKIEELFKTFFGSIKSMWKHSPISGSESSPQSSTPDNIITDKLDDQDRRLKDQGDSENSSLFGLKLPSFCKVN

SEQ ID NO: 9 - protein, 128 aa, unidentified
FRSLKIGRKQSTAVKGVLTCNGKPAVNVKVKLYNDSQGRYVENSMDEGKTDSEGRFLLQGHETSITSIDPILKLYHNCDVENAQCLKRFSILIPNDFVSEGLEPKKTFDMGTLNLGGKFFDEGRECAS

SEQ ID NO: 10 - protein, 256 aa, unidentified
QIIGSFNGNYAGDGSLNNNANSFGERTTTTRSTSRPSLPPRPGYPSRPGYPFKPGFPPRGPPIPYPHGKPSGPRYPCYGGYGGYGHPGYGPFGGNGYLGYTVCSGRGEFGGYGPGLGGGTGLGGLGPGEFGGYGPGLGGGTGLGGLGPGGFGGIGPGLGGGGGLGGPGRGGFAGYGPGLGGGRGLGGPGPGGFDGYGPGLGGRPYPGGYGRFYGPGPYPGDRLDPRGLSESGRPRTRLASYNRNDRGTQFSYIRDR

SEQ ID NO: 11 - protein, 280 aa, unidentified
MTNEYETNYPVPYRSKLTESFEPGQTLLVKGKTAEDSVRFTINLHNTSADFSGNDVPLHISVRFDEGKIVFNTFSKGEWGKEERKSNPYKKGDDIDIRIRAHDSKYTIYVDQKEVKEYEHRVPLSSVTHFSIDGDVLVTYIHWGGKYYPVPYESGLSGEGLVPGKSLLIFATPEKKGKRFHINLLKKNGDIALHFNPRFDEKAIVRNSLIAGEWGNEEREGKMILEKGIGFDLEIKNEEYAFQIFINGERYATYAHRLDPREINGLQIGGDLEVSGIQMR

SEQ ID NO: 12 - protein, 141 aa, unidentified
DLLSEAGDFFTKHFTDIKSLFAKDEKQLQQSVDRVKDLLATIQDKMSMLQPLANDMQKTTLGKIGDLISQVNSFRETMSNPKMDFTNKENKWEELLKKIFVTEGLNKVIPLLQKLKNSAPTTFATYLFTCIVPVLINTLRE

SEQ ID NO: 13 - protein, 114 aa, unidentified
MARINRLNFLLCIVHANITSAPNPKDSNDELFADAESALGSEYAQFVEQSKQHKPVYFSDNQNTLETIKLESIPNPETETAYPMFICGFLGCMKKMNSVEEYLEHFKMHEKQGY

SEQ ID NO: 14 - protein, 69 aa, unidentified
FSWKFGERLDEPVLMLRDLRAKEISPPSYMKRFESDTNEQLLRYILHPKMLRRHDLSNALFYQPLWKMR

SEQ ID NO: 15 - protein, 278 aa, unidentified
MEGSPIKETRGLEATPVFEMVRSATLTFLLAVSTVLVVSRPNVLLPPKLPWDSDWRQKPPPFPPEPPPEFKGILPPEIFAKLTAIHQDQSLTIPQKIVKIEEIMNSLPEDVLQRLPLPPVFRLLPQNVQEMIKTVRTTKNLTMEEKWLQMIILIESLPKQQHRLLQQMLPKFSLGPLPDFQDIIPKEDWDKLTAVYQDTNLDNIEKLRRVDEIIDALPDSIRQKIPLSPPFQKLPDHIQQQLQIIHTERGLTTEQRFRKMKAIIESLPWDMKKLMFQP

SEQ ID NO: 16 - protein, 425 aa, unidentified
LIKVFPEISANMSVMFANSRSNQANNGYLVEFKAGRSNLQAGSTVDRRKVVADKTKGLVFIKQSSDQLMHFCWKNRETGAVVDDLIIFPGDTEFLRVRECTDGRVYMLKFKSTDEKRLFWMQDGKTDKDDENCKKVNETLNNPPAPRAAARGGADRADVSSFGTLAALGSAGAESELGALGNLDQSQLMQLLSLMNHTNSTSASEATNLLPQLPLVADTSHPMTSEDSGTTSTHGATPSNTPANGIVADSSSNNAMQLSQLKEIIASITPPDGSGRKPSIDFTDVLCCADKINDVLRKYAEQLIPHLPSQEPIYNNQEELQQTLRTPQFRQAADIFGHALQTGQLAPVLRQFGIDGNTATAAGNGDMVAWAAQFTTAENGKEITAKTETSPSQPGMESDVEDEETNEKAIRETEKNRTDDHMDLD

SEQ ID NO: 17 - protein, 90 aa, unidentified
MNYKAPIELQQLLSITKMLSLSVLLLFTSMAIMARPPNSDEIKELRQQQLNESKDDYDTLPDVNHIPESFKESLKKQKMLYLDMLRQHNL

SEQ ID NO: 18 - protein, 257 aa, unidentified
LSVPAGLRPAKKVGDPKEQIVPGKQQQLQQQQQQQLLQQQQQQQQQQQQQQQQQQQQQQQQQQQQQQEQQEELQQLQESEETGEEHRQQQQQQHDEALTLSPTPKVPPNLSIRSRMMAALSASVGESNKEKNSSNDETDNSSKSTNSPSKPPIIFPKANKKTVVGKIAPSGISKGSARVIVAPPSKLGTNNFGLNTVLQTNLVDSRGRIMKNVNSVPIKVPSSAEMRNARTRHTARQVESDADKVVPIKFGSTSRRR

SEQ ID NO: 19 - protein, 130 aa, unidentified
NNSNLDISMREKNAVNAIEKQDLPRSHRFKRQYSCGQCGGGGGPPVVVSPCQQCKGGGAGVSAIGGAGGISAIGGGVSAIGGGFGGGGGDTVAVVCCGATGLKGMFRNWWLHIPLLLLPMSMSWIKALFL

SEQ ID NO: 20 - protein, 185 aa, unidentified
YYVPDNYWPLRIIGYHHIPVMINMWYLFQTEISNIGVDAVLVQSPLYRTLTPDVVHDIISINVEPNHTVVVEQSNPMLQASSVEQAPAAAPLSITLIAPGITISRTHKVDTYKSTMEMYDADKLHSNEIFKRRVRKMVLPPSRGEEVRKPPSSTDGYESENVESYGQKGVEQAPPEIEQYVKKKK

SEQ ID NO: 21 - protein, 633 aa, unidentified
MKYCLSSIIAATIATTTTTATAIIATTITAATISVAPFHASSPSSSLSSSSFSSFFLVLPLITTILLIVPEQAHSTATVTEHRSPPDLSIPSQTEFRVPVGTKQFRLICPVKEKNDDLLMIQWKKNDEPIGFDFNNRFKLARSDRELKIRNPQLSDGGIYQCQVVNGFGHRELNFTVTFYDPAMENDQNTDSTLTLTTKASPPIWKNETEIRNWMINPVRITIGGALLLKCPAKGNPLPHITWLRDGKVLEREITYHYSSAILYLSDVQPSEGGKYICKLENEHGSIEASFHVYVENFFEGLDGESWSIDQTNAQLYPVIDEPFNNTVRVGRTAQFQCKVKNQQQPLIKWLKRVEDPNAIRQTNANATLIHANNMHLLLLEKPETSAELSDGISLNRLIIPNVRYEHSGTYLCVVTNARGDIAYRSAYLNVIARSDHGELSNLYFYGGLLVLIVVFTLITYAVHFLRKNQAAKSTESAPGITNIRYSFSLRPPPPNLPPPKAPALPSERQQLMPNNQPCDRYTVNSAAATYYPQFATPDKKLQKIITESGTRPTPIRRTNGGDTKYRLKDDYISSPKWVHAKGDNIEVEMDQNLLKNRSTHCHNPVSIAYGRIDNIDRQQQKSFLTIGNLQKR

SEQ ID NO: 22 - protein, 189 aa, unidentified
KEIIWDCYGDYEECVAESSKMDHVDVNNVESRNIIEFCSDHTQNILPCLATKLGLIKSMSVSMFSLLLTICEAETRNNRPAATEVQQILKHLARLYAYFCAYSNVIDLRYNKECFRYLKKRCILNKPDDSCIFHHCGEKNLNLSESSPFIQQHKTTIINQLNQSATFKNYHHRITTIFTVIITFISMIQ

SEQ ID NO: 23 - protein, 399 aa, unidentified
MYNQENHDKRRNDDRFILSLPFGTNVENKSYFKPIKLSNPYSDKYLEVNKKSSDDSDQNLNQALSVPQSNYDQSSESLSIDDSDLIDDSTSAAQLSTSSPISVTSASTSSFYPTLNIGNGMEISAKYAKLEQSQGIKSDQSTSRVSDRYKKYTAVKRRLSELYGIIEEKDEQLRVVRNELNGKDLEIGKLCDKIRALEYNCGRLQSMIESAGDESDQNQVKLHEIINERDGLLIRNASLSRQIEFEKREWSIERERLSMDLDDVTRELELQKMILNGESISEIVQRWQTKVFELEGMITDRDRAIRAQQVQISKLKESIAETDRISCADSSESQTKFDFPSFTYIKRLLLQYLTRLADLHFSSDEERMQLVRNMSSILHLSDEEQRQVWANLKSKIQIS

SEQ ID NO: 24 - protein, 301 aa, unidentified
QCPTGSVSLLSGYRCTSSIQCQTIIPGSYCYYGVCCTGGSDVLSKTVSYGGYCTMTVQCSTTGATCISNICQCDINSHYNGHSCVSISNFCPSNQVFIKGECYRKVTYGFLCNYTQQCGYIGAFCIGNICSCQLDYTFDGSKCIPRSRICPANQIAIGGQCYPSARFGERCLYSEQCIDRWYRSLSCVNGFCNIRNDDDISKPKCRNPRAEVEYVNGTAKNCLYWPCTVGYFCEYAGGMNGGRYICCGTNANKIYGKVQLYPGTGTPLQCTEIGRCPFPDTPNCVMSYRYGYKVCCSTLNC

SEQ ID NO: 25 - protein, 110 aa, unidentified
QETSEQPGLTVEIIAEQQDATTADQEVTTTVDTHHQHQHQTDKVVKSRQITGDEQTTTTTTAINLNETITNSTTDSNSTIITTTLDLQESTTTGTTDNHHHHHHHHHHHE

SEQ ID NO: 26 - protein, 185 aa, unidentified
MKQTTAWGNALCVLCNCHQPQIICPPPPPAVCPRVVCPPPRPPVCPPIYCPPPVVCPPPPVCPPVPFCHSQICPPCGTHTVPVAVVGCCKGCACSVRFKRDSSSVNGLMLKKNLLCNNDQLMTIMEKKIGTNATEAAFAIKKEADSELKAKFSVFCAMNDLIYVAHAESFCQHKKGDIICFAYKS

SEQ ID NO: 27 - protein, 147 aa, unidentified
MDCKLILPFYILLANLEANAFHLSGYRSRSYLQGIQPYDIQPLDVQPQFIRVQTLKSQDIQPYSIQSRSEDQPCEGCKITISCGSKNCKSKKLPYVYKPIFKLLSTRSTKKPVFTLPTQPPAQWDCPCPCHVPQRCRMCSACHESYI

SEQ ID NO: 28 - protein, 194 aa, unidentified
NRIISRRLSLFIQQYCCNNISQIYRLNDCKYSKVKMEIDKKIFIIVSKTEWCNEAIKVVFGKSAEAIRNNSDAISWLASYNYTGSMDLRSKWPYDAYFDNVTRTAHGLARIDLLCHKKRPQLGPRIWKRSVQKIKQKKDRPFAVNTYGNNKGLFTITVGVLLYAAFGTCFLIANLAYLFGIYIIYDASIIDEVS

SEQ ID NO: 29 - protein, 263 aa, unidentified
IGENPMDVNAIAGIIGGISNMMQNNVETIDVPSSQIMGRWYQVYKAAIAFDVYRTDIFCPVAYFKPNSVMGEDGFSIEEAYRVITKNGPVETYKRDLNKVGTGQYWMYTEEYFYPRQFNIISVGPNYTNTTDGSEEEKQYQYMVVTDGNRLSLSVYARHPMIFYQKYNEEVVKFLEHAGFGGKVFWNSPKPIYQGADCEWPSEKEVFARRVLKNQELAKNGGLDTATKSGSFGGSSQATDVRSSITEILQNPQLALQKLVQGH

SEQ ID NO: 30 - protein, 242 aa, unidentified
MTIIKSMLKITHVIFDLDGLLIDTEVVFSKVNQCLLSKYDKKFTPHLRGLVTGMPKKAAVTYMLEHEKLSGKVDVDEYCKKYDEMAEEMLPKCSLMPGVMKLVRHLKTHRIPMAICTGATKKEFEIKTRHHKELLDLISLWVLSGDDPAIKRGKPAPDPFLVTMDRFKQKPEKAENVLVFEDATNGVCAAIAAGMNVVMVPDLTYMKIPEGLENKINSVLKSLEDFKPESVGLPAYDASSNE

SEQ ID NO: 31 - protein, 148 aa, unidentified
IPQRRQQQQQQQQQQQRDEREIPPFLEGAPPSVIDEFYNLLKTDENKTDQQTEADVEAFINRLGGSYKVRFTQFMEEVKKARADYERIHQQAVARFSPAAKDADARMSAIADSPHLTTRQKSQQIQAIMDSLSESVRREIINALSPQE

SEQ ID NO: 32 - protein, 141 aa, unidentified; identical to SEQ ID NO: 12

SEQ ID NO: 33 - protein, 446 aa, unidentified
MRTASQLTFMLFLVLKKKFKNIDKLFSQISVNMSVMFANSRSSQANSGYLVEFKAGRSNLQAGSTVDKRKVVADKTKGLIFIKQSSDQLMHFCWKNRETGTVVDDLIIFPGDTEFLRVKECTDGRVYMLKFKSTDEKRLFWMQDGKTDKDDENCKKINETLNNPPAPRAAARGGADRAGASSFGTLAALGSAGADSELGALGNLDQNQLMQLLSLMNHTNSASASEAANLLPQLPLVADTPNPVASEESGTTSTQGATPSNTPANGIIAGSSSNNAVQLSQLKEIIASITPPDGSIRKPSVDFTDVLCCADKINDVLGKYAERLIPHLPNQEPIYNNQEELQQTLRTPQFRQAVDIFGHALQTGQLAPILRQFGIDSNTAIAAGNGDLIAWATQFTTSENEKEIAVKTETLPFHPGMESDVEDEETNEKAVRESDKNRTDDHMDLD

SEQ ID NO: 34 - protein, 114 aa, unidentified
MLPTLYINNAVIRPVLSETKKVKVQNISSPFLIFLLLSITKMLSLSVLLLFISMATMARPPNPDEIKELHEQQLNDSKDDYDMLPDVGHIPESFKESLKKQKMLYLDMLRQQSL

SEQ ID NO: 35 - protein, 298 aa, unidentified
MISSRLRITIPESIVIFGIFCFFIFFCFLSFFFFFTLWSHRDTINFQTDFMTETIKFIVYAVVILRMMFFDIVCFYSFLMMTIVLINTSNGLSVPAGLRPAKKVGDPREQIVPGKEQQQQREQQQQQQQQLQEEEQQQQQQHDEVSNLRPTPKVPPNLSIRSRMMAALSASPVEPNKEKNSSKVETDSFSKPPIIFSKGNKKTVPGKIAPSGSSKGNARVIVAPPADLGKNNYGLNTVLQTNLVDSHGRIMKNVNSVPIKVPSSAEMKNARTRHTARQVESDADKVVPIKFGSTSRRR

SEQ ID NO: 36 - protein, 99 aa, unidentified
MMRIKWIILLLLLLLPIITAEFSAPVGTNSSLTIFDKDKQVLLRSDRLKRQCGPCGVAPSPVIVCCGAAGLKEIFRSWWLHIPLLLLPMSTSWLKTMVC

SEQ ID NO: 37 - protein, 193 aa, unidentified
MFRLLIAIQILRFCQANYINDVYWKRSIIGYQHIPIILNICYLLQTEVSNKGVVDALFLHSPTYHRVEMSEETDNIESIADKSNITVANKPNLMIYPADFQVSSNERASASIPITITITSSGDTIIKSFKHKHQSNEIFKRRVAKMAIAPVNAPEVENLAPEVENPSPSTAGYESKTEEQAPSESGQYGKRRK

SEQ ID NO: 38 - protein, 362 aa, unidentified
MYNLAKLLENEHGSIEASFHVYVENFFEGLDGESWSIDQTNAQLYPIIDEPFNNTVRVGRTAQFQCKVKNQQQPLIKWLKRIDDPNAIRQANANATLIHANNMHLLLLEKPETSAELSDGISLNRLIIPNVRYEHSGTYLCVVTNAHGDIAYRSAYLHVIARSDHGMLSNIYFYGGILVLIVVFTLITYAVYFLRKNQAAKNSESAQDITNTRYSFSLRPPPPNLPPPKAPALPSERQQLMSDNQPCDRYAVNSAATTYYPQFATPDKKLQKIITESGGTRPTPIRRTNGGDTKYRLKDEYINSPKWVHTKGDNIEVEMDQNLLKNRSSHCYNPISGAYGRTDNIDRQQQKSFLTIGNLQKR

SEQ ID NO: 39 - protein, 215 aa, unidentified
MLKLANTEIFFIAFLVYSKEIILNCYEDYKECVATSNKTNHVNMDNVNPQNLIEFCFDHTQNILPCLVTKLGLTKGISVSIFSLFLSTCELEAQNNKSSSTTEMQQILRHLLRLYAYFCAYSNIIDLHRNRECFRYLMKRCVLNKPDESCMFYHCGKIHFNLSKSSRKILFTRQHDTTKIVNLGNKMNQLATFNNHQVRSAVVVTLIITFIDMIQ

SEQ ID NO: 40 - protein, 395 aa, unidentified
MYSQENQDDKRRNDERIALSVPYNNTNIMDRSYFKPIKLSYPYSDECLEVNKKSSDDSDQRLSQNSSTPQSNYDQSSERLSIDDSDLIDDSTSAAQLSTSSPISVTSASTSSFYPTLNIGNGMEMNAKYAKIEQSEGIRSDQSSTLRISDKYKKYTAIKRRLSELCGIIEEKDKQLRVVRNGLNEKDLEIGKLCDKIRALEYNCGRLQAVIESVGDESDQNQIKLHEIINERDGLLVRNASLSRQIEFEKREWSIERERLSMDLDDVTRELELQKMILNGENISEIVQRWQTKVFELEGMIADRDRAIRAQQVRISKLKQSLAEADRISCDDSSESQTKLDSPSFTCIKRLLLQYLTSSDEERIQLLRNVSTMLHLSDDEQHQVLTNLKSRIQIS

SEQ ID NO: 41 - protein, 99 aa, unidentified
  SITE 1: note = misc_feature - Xaa can be any naturally occurring amino acid
XKCRDQRAEVEYVNGSAKNCLYWPCTVGYFCEYTESRNGGHYICCGTNANNIYGKVKVYPGTNKPLHCSIMNTCPFLDTPNCVMSHRYGYKVCCSTMNC

SEQ ID NO: 42 - protein, 589 aa, unidentified
MLMKQSDSCVDYFYDQYKGQEYVKDDAFNTQNITDNFRKSSSDIAQLMNSQIELISQPEKVNEDSAKSSHYNDDLQKSIEDDTVEATQRKKDEKLLEFLHSLIVSTIPKTIHLEGNSVNLLTLTTTITPIAIITTKNTSGTANAITTRKYKKYKLNAFVNISSDTLTELPKFLPENFNSTNFANVEKTEKFSNSKQVATDSIFSLKESAYLETPVIRDFSSANDSAKTDPLFTRNYVDKQIDMNTTKFNKNLKKSRLTTISTSNLTTVLSQLQTTTSISTTTSVTTTISTSITIPELTLVSQSHRHLHHYHHHHHHQYENYDHESPIIVTALFDIGRGKWPRYTRTYEQYMNYLKHLLKLENCLVIYTDSRGAEFVRQTRNVHNTQIFEISMHDLPLYRYREEMKGIIQREQKDWQFSPKTRYHPEANSADYNIIVNSKPYFLYNATQNVRFRTSDRMFVWIDAGYGHGRKGIIPDHCHWRPRLQRDRMTIIQLTPKHDKVSRYSITDLYRVDWVVLSGGFIAGDSHTINRFYRFYQKLFMELLDSGRIDDDQTILTLMLKHYTTLFNPISSNGDWYALFRLFPCHDRQ

SEQ ID NO: 43 - protein, 875 aa, unidentified
MKQATTWGSICEMCPCAAKPICPPPVICPPRICPPPVICPPQICPPCPPRICPPPVICPPQICPPCPPQICPPCPKPQPPPPPPPPPVLPSLPPTSFKPMITCCRTCICYIRRKRDSLNDYDRIHDINPVCNNDQLMMIMKKKIRTNVTESTIAIKKAADSMLQAEFNVFCAINDLTHVAHAEHFCQYKKDNSVFDSFLFRSTLKGLIEECREGVRWWPGSLGDLDFSHISLYRAHKYIGNEEMNRSTKTKISFTRINKKWRLGHTGKKYNKVRFSRNIAKKFIGVCNIIRLKKSVSRSVRPFENQKSTSFNVFQLLVPKEKVEIVVDDTQAEEMNSETAQEVQLFNVRKSNADSKTDGEKDTADLDVILLTNEECSSSRQENLNKDEPEIVILDDSAPSKSDLNTSDEIICLQDLKMVNEVPTFSVTPKQKTVKELPRETRTYGTRRGRQSRAYCEDLRKFPSIRNPVSSSSSSIHAKNMPEFVDLLTQGTLLICKKWLRRWDIVQSGVIGGNPLRICSYNVLCQQTAYKTPELYIHLTKPGRAYELTWENRWRLLTREFSMIGADIFCLQEVQYDHYDQFFRPYFEAAGFFGKYKKRTNNLLDGCAIFYKSHLQLLHYRYIEYFLNIDSVLNRDNVGQLIRLKDMRSGREFCVVNTHLLFNKRRGDVKLAQLAILLANIDQECGPESGQECPYILCGDFNFHPYSPIYNFIMNGEICFTNLRRGDISGQGNAGGPFVSVNLLPEDVKIARNCRFNYLKNRTMLLPSLNCWSHPLCFNSVYQNMNGETRPMISTYHSIEAVNPDFIFYSVKSKRVQQSMLPHSVPAMNVSEREIRLIRRLSLPDMNELAGTLGPWPNSTTPSDHIPLIADFVLQ

SEQ ID NO: 44 - protein, 158 aa, unidentified
MYCKLIISFYMLLSIANMTHLVGYRPQIYLQGIPQNIQSHDIQRLDMQQQSLKLPDTELYSIPSHDNQLQGLQLYDMQFQGKQSKGSEKLCSGCKISINCSGKKCVPMRTRKPIVTTPSPLSTQRPVLTRPRLLADCPCPCHVSRQCRICQPCQESFI

SEQ ID NO: 45 - protein, 230 aa, unidentified
MFVGMRLYLAIDVLLLLVLRIKSNRIILHRFSLFIQQHCCNNISQIHRLNDCKYSKVRMKIDKKILIIVSKTEWCNEAIKVVFGKSAEARRNRSDAISWVTPYNFTGLMNLHSKWRYDAYFDNVTRTAHGLARIDLLCPKRRSHSGRRILKRSIQENKQEKSRRSFTVNIYGSSKGIFTITVGVVIYAIFGVCFLITNMAYLSGIYTVHNTSVIPEDKKRKETSKRKEIL

SEQ ID NO: 46 - protein, 279 aa, unidentified
MISVFLLLTVIVSYVETIGENPMDINALAGIIGGISNMMQNNVETIDVPSSQIMGQWYQVYKAAISFDAYKTDMFCPVAYFKPNSVMGEDGFSIEEAYRVITKNGPVETFKRDLNKVGTGQYWMYTEEYFYPRQFNIIGVGPNYTNATDGREKENLYEYMIVTDANRLSLSVYARHPMIFYQKYNEEVVKFLEHAGFGGRVFWNSPRPIYQGTDCEWPSEKEVFARRVLKNQEAARNTGLETATKSGLFGSSLTTDAYNPIKEMLQNPQLALQKLVQGH

SEQ ID NO: 47 - protein, 200 aa, unidentified
MTVIKSMLNITHVIFDLDGLLINTEIVFSQVNQCLLSKYGKKFTSHLRGLVTGMPKKAAVAHILEHERLSEKIDVDEYCKKYDEMAEEMLPKCSLMPGVMKLVRHLKAHSIPMAICTGATKKEFELKTRCHKELLDLISLRVLSGDDPAVKRGKPAPDPFLVTMERFKQKPEKAENVLVFEDATNGVYAAIAAEESKIVK

SEQ ID NO: 48 - protein, 116 aa, unidentified
MILEQLEVPPFLVGAPQSVIKQFYDLLKADETKTDAQTEADVEAFINRLGGTYKTRFDQFKQEIKQGKAAYERLHQQAVAKFSKEAREADAKMSAIADSPSLTTQQKTQQIQAIMD

SEQ ID NO: 49 - protein, 158 aa, unidentified
MLKYGILLILITVGAYCDLLSEAGDFFSKHFTDFKSLFASDEKQLQQNMDRVKDLLATIQDKMTILKQLADNSQKSTLEKITDIISQVNDFRENVFNSNVDFNQKKTKWEEVVTKIFVTDGLNKVIPLLQKAKNSAPATFITYLLTCIVPLLINALRE

SEQ ID NO: 50 - protein, 20 aa, unidentified
  REGION 1..20: note = Flexible linker
KGPDVPETNQQCPSNTGMTD

SEQ ID NO: 51 - protein, 114 aa, unidentified; identical to SEQ ID NO: 13

SEQ ID NO: 52 - protein, 20 aa, unidentified
CPSLSSYCEDWDPEDFPSFV

SEQ ID NO: 53 - protein, 29 aa, unidentified
LPIDKQNEATLLWNSLYPDDIYNECGPRF

SEQ ID NO: 54 - protein, 25 aa, unidentified
AFAPNPKDSNNELFADAESALGSEY

SEQ ID NO: 55 - protein, 24 aa, unidentified
GCMKKMNSVEEYLEHFKMHEKQGY

SEQ ID NO: 56 - protein, 36 aa, unidentified
INKFYDMLRKWAEKYSVQAETNRFIAEEMNYDKMQS

SEQ ID NO: 57 - protein, 35 aa, unidentified
APPNRDTADDLQNADMQRQWEQEQRQREEVQKEEI

SEQ ID NO: 58 - protein, 66 aa, unidentified
TDMDELEEQRKQDFKQYEMKKKAEEDHKMQAIQTEREEYIRQMEEQRRRHNKHEPLKHPGSRNQLR

SEQ ID NO: 59 - protein, 28 aa, unidentified
DREAQEEKPDQGWEDIGDKDQYTKEELE

SEQ ID NO: 60 - protein, 30 aa, unidentified
TPAPTPDPSRMIQPDQAPMQRLDAPSDQVG

SEQ ID NO: 61 - protein, 21 aa, unidentified
VSEILTVCLKEGFKKPSNLGI

SEQ ID NO: 62 - protein, 130 aa, unidentified
KNPSKMESKTGENQDRPVLLGGWEDRDPKDEEILELLPSILMKVNEQSKDEYHLMPIKLLKVSSQVVAGVKYKMDVQVARSQCKKSSNEKVDLTKCKKLEGHPEKVMTLEVWEKPWENFMRVEILGTKEV

SEQ ID NO: 63 - protein, 181 aa, unidentified
KISAENANCKKCTPMLVDSAFKEHGIVPDVVSTAPTKLVNVSYNNLTVNLGNELTPTQVKNQPTKVSWDAEPGALYTLVMTDPDAPSRKNPVFREWHHWLIINISGQNVSSGTVLSDYIGSGPRKGTGLHRYVFLVYKQPGSITDTQHGGNRRNFKVMDFANKHHLGNPVAGNFFQAKHED

SEQ ID NO: 64 - protein, 436 aa, synthetic construct
  REGION 1..436: note = Ov-103-RAL2-CPI2M fusion protein
DLLSEAGDFFTKHFTDIKSLFAKDEKQLQQSVDRVKDLLATIQDKMSMLQPLANDMQKTTLGKIGDLISQVNSFRETMSNPKMDFTNKENKWEELLKKIFVTEGLNKVIPLLQKLKNSAKGPDVPETNQQCPSNTGMTDPQRRQQQQQQQQQQQRDEREIPPFLEGAPPSVIDEFYNLLKTDENKTDQQTEADVEAFINRLGGSYKVRFTQFMEEVKKARADYERIHQQAVARFSPAAKDADARMSAIADSPHLTTRQKSQQIQAIMDSLSESVRREIINALSPQEKGPDVPETNQQCPSNTGMTDKNPSKMESKTGENQDRPVLLGGWEDRDPKDEEILELLPSILMKVNEQSKDEYHLMPIKLLKVSSQVVAGVKYKMDVQVARSQCKKSSNEKVDLTKCKKLEGHPEKVMTLEVWEKPWENFMRVEILGTKEV

SEQ ID NO: 65 - protein, 286 aa, synthetic construct
  REGION 1..286: note = Ov103-RAL2 fusion protein
DLLSEAGDFFTKHFTDIKSLFAKDEKQLQQSVDRVKDLLATIQDKMSMLQPLANDMQKTTLGKIGDLISQVNSFRETMSNPKMDFTNKENKWEELLKKIFVTEGLNKVIPLLQKLKNSAKGPDVPETNQQCPSNTGMTDPQRRQQQQQQQQQQQRDEREIPPFLEGAPPSVIDEFYNLLKTDENKTDQQTEADVEAFINRLGGSYKVRFTQFMEEVKKARADYERIHQQAVARFSPAAKDADARMSAIADSPHLTTRQKSQQIQAIMDSLSESVRREIINALSPQE

SEQ ID NO: 66 - protein, 297 aa, synthetic construct
  REGION 1..297: note = OvRAL2-CPI2M fusion protein
PQRRQQQQQQQQQQQRDEREIPPFLEGAPPSVIDEFYNLLKTDENKTDQQTEADVEAFINRLGGSYKVRFTQFMEEVKKARADYERIHQQAVARFSPAAKDADARMSAIADSPHLTTRQKSQQIQAIMDSLSESVRREIINALSPQEKGPDVPETNQQCPSNTGMTDKNPSKMESKTGENQDRPVLLGGWEDRDPKDEEILELLPSILMKVNEQSKDEYHLMPIKLLKVSSQVVAGVKYKMDVQVARSQCKKSSNEKVDLTKCKKLEGHPEKVMTLEVWEKPWENFMRVEILGTKEV